(19) United States
(12) Patent Application Publication
(10) Pub. No.: US 2022/0163429 A1
(43) Pub. Date: May 26, 2022

(54) NON-CONTACT CELL MANIPULATION SYSTEM, DEVICE AND METHOD

(71) Applicant: New York University in Abu Dhabi Corporation, Abu Dhabi (AE)

(72) Inventors: Mohammad A. Qasaimeh, Abu Dhabi (AE); Ayoola T. Brimmo, Abu Dhabi (AE)

(21) Appl. No.: 17/534,718

(22) Filed: Nov. 24, 2021

Related U.S. Application Data
(60) Provisional application No. 63/117,748, filed on Nov. 24, 2020.

(51) Int. Cl. G01N 1/10; C12Q 1/6806

(52) U.S. Cl. CPC G01N 1/10; G01N 2001/1006; C12Q 1/6806

(57) ABSTRACT

A non-contact cell manipulation system comprises a supporting member, a micro-positioner, a probe holder having a proximal end and a distal end, the proximal end connected to the micro-positioner, a probe adapter removably connected to the distal end of the probe holder and a non-contact multiphysics probe fluidly and electrically connected to the probe adapter, wherein the probe includes at least one electrode, at least one aperture, and wherein the probe is configured to utilize electropermealization and electroheating in combination with hydrodynamic flow confinement to perform non-contact cell manipulation. A non-contact multiphysics probe and non-contact cell manipulation method are also disclosed.

### CROSS-REFERENCE TO RELATED APPLICATIONS

This application claims priority to U.S. provisional application No. 63/117,748 filed on Nov. 24, 2020, incorporated herein by reference in its entirety.

### BACKGROUND

The heterogeneous function and spatial arrangement of individual cells is critical to the identity of the host multicellular organism. While single-cell manipulation and analysis techniques are now capable of resolving spatial heterogeneity, they mostly rely on extracting the target cell from its physiological environment. The ability to manipulate single-cells within a tissue sample, while retaining their spatial arrangement for time-dependent cell developmental analysis, will facilitate a better understanding of cellular networks.

Such heterogeneity is increasingly being appreciated in the analysis of complex diseases like cancer since therapeutics developed to target one tumor cell phenotype is not necessarily effective against another. Based on this, individual cell ‘omics’ parameters are now the benchmark for a true systems-level understanding of how multicellular organisms function. The classic methods for isolating single-cells are micromanipulation, laser capture microdissection, and fluorescence-activated cell sorting (FACS). In the last few decades, microfluidics has been turned to as the technology of choice for single-cell isolation as it offers integrable platforms for multiple cell manipulation operations. Advances in this field include the isolation of individual cells using droplets, micro/nano-wells, valves, and di-electrophoresis. These adaptations have led to the uncovering of important cell biology insights like the principle of DNA regulatory variation, resistance-linked genetic expressions within melanomas, complete lineage hierarchies of the developing lung, distinct stem-like gene expression signature of early-stage metastasis of cancer, and the function of mobile genes in human microbiome. However, while the spatial arrangement of cells is vital for tissue function, most of the current single-cell methods rely on procedures that dissociate cells from the tissue samples and cannot retain the original cell organization. Hence, a robust comics' profiling of a process that is inherent to the cellular environment (e.g., gene expression) still remains a challenge for the available techniques.

### SUMMARY

In one aspect, non-contact cell manipulation system comprises a supporting member, a micro-positioner, a probe holder having a proximal end and a distal end, the proximal end connected to the micro-positioner, a probe adapter removably connected to the distal end of the probe holder and a non-contact multiphysics probe fluidly and electrically connected to the probe adapter, wherein the probe includes at least one electrode, at least one aperture, and wherein the probe is configured to utilize electropermealization in combination with hydrodynamic flow confinement to perform non-contact cell manipulation.

In one embodiment, the probe adapter is removably connected to the probe holder via a twist and lock connection. In one embodiment, the probe adapter and the probe are fabricated as a single piece. In one embodiment, the probe is connected to a robotic arm for linear positioning control in XYZ and rotational positioning control around X, Y, and Z axes. In one embodiment, the probe is connected to an atomic force microscopy setup. In one embodiment, a subset of the at least one electrode is configured as a counter-electrode. In one embodiment, the probe is a 3D printed part. In one embodiment, the system further comprises a cell culture glass slide coated in an ITO substrate. In one embodiment, the electrode and the ITO coated substrate form a pin-plate electrode. In one embodiment, the ITO coated substrate is configured as a counter-electrode. In one embodiment, the probe comprises at least two electrodes, where one is a working electrode and the other is a counter electrode. In one embodiment, the probe comprises both working and counter electrodes and can work with any nonconductive substrate supporting biological samples. In one embodiment, the probe and its manipulation setup are configured to work with adherent cells. In one embodiment, the probe and its manipulation setup are configured to work with tissue slices. In one embodiment, the probe and its manipulation setup are configured to work with tissues in vivo. In one embodiment, the probe and its manipulation setup are configured to work with neurons. In one embodiment, the probe and its manipulation setup are configured to work with genetic material. In one embodiment, the probe and its manipulations setup are configured to work with proteins. In one embodiment, the probe comprises at least one working electrode and at least one counter electrode. In one embodiment, the probe comprises fluidic networks, chambers, filters, droplet generators, multiphase systems. In one embodiment, the electrodes are integrated within the probe's bulk for heating, sensing, and pumping. In one embodiment, the capacitive electrodes integrated on the probe's surface for micro positioning control.

In another aspect, a non-contact multiphysics microfluidic probe comprises at least one aperture, and at least one electrode, wherein the electrode and aperture are configured to perform electropermealization in combination with hydrodynamic flow confinement. In one embodiment, the probe is configured to perform electropermealization. In one embodiment, the probe is configured to perform electroporation. In one embodiment, the probe is configured to perform electrolysis. In one embodiment, the probe is configured to perform electroheating. In one embodiment, the probe is configured to perform electrosensing. In one embodiment, the probe is configured to perform chemical stimulation. In one embodiment, the probe is configured to perform electrical stimulation. In one embodiment, the probe is configured to perform biosampling. In one embodiment, the probe is configured to perform dielectrophoresis. In one embodiment, the probe is configured to perform combination of electropermealization, electroporation, electrolysis, electroheating, electrosensing, chemical stimulation, electrical stimulation, biosampling, and/or dielectrophoresis.

In one embodiment, the at least one electrode has a radius of 0.1-100 μm. In one embodiment, the at least one electrode has a height of 0.1-1000 μm. In one embodiment, the at least one electrode is at least one of a dome shape, a conical shape, a spiked shape, an arc shape, a pin shape, a cylindrical shape, a pyramidal shape, and/or any shape with converging/diverging diameter/width across its length. In one embodiment, the probe comprises at least one aperture used for injection and/or aspiration simultaneously and/or sequentially. In one embodiment, the probe comprises at least one inlet aperture and at least one outlet aperture. In one embodiment, the at least one inlet aperture has a radius of 0.1-1000 μm, and the at least one outlet aperture has a radius of 0.1-1000 μm. In one embodiment, the at least one inlet aperture and the at least one outlet aperture are spaced at least 1 μm center-to-center apart. In one embodiment, the at least one electrode is positioned centrally between the at least one inlet aperture and the at least one outlet aperture. In one embodiment, the electrodes are positioned in line with the at least one aperture. In one embodiment, the electrodes are positioned diagonally to the apertures. In one embodiment, the electrodes are positioned perpendicularly to the apertures. In one embodiment, the electrodes are positioned external to the apertures. In one embodiment, the electrodes are positioned internal to the apertures.

In another aspect, a non-contact cell manipulation method comprises providing a non-contact cell manipulation probe, positioning the probe above a cell to be manipulated, applying an electric field via an electrode located on the probe, and applying hydrodynamic flow confinement to manipulate the cell.

In one embodiment, the electric field and hydrodynamic flow confinement are applied while the cell is still within its original tissue organization. In one embodiment, the electric field and hydrodynamic flow confinement are applied while the cell is adherent to culture plate. In one embodiment, the electric field and hydrodynamic flow confinement are applied while cells in round shape. In one embodiment, the electric field and hydrodynamic flow confinement are applied while cells are mechanically trapped. In one embodiment, the electric field and hydrodynamic flow confinement are applied while the cell is captured on surface. In one embodiment, the electric field and hydrodynamic flow confinement are applied while the cell is still within its original 3D tissue in vivo. In one embodiment, the electric field is pulsed. In one embodiment, wherein the manipulation performed is a single-cell biopsy operation. In one embodiment, the manipulation is performed to target sub-cellular components. In one embodiment, the manipulation is performed to target single cells. In one embodiment, the manipulation is performed to target a group of cells. In one embodiment, the manipulation performed is a heat assisted single-cell tweezer operation. In one embodiment, prior to positioning the probe over the cell, the probe is zeroed based on horizontal displacement when the probe contacts a substrate. In one embodiment, the method further comprises positioning the probe 0.1-1000 μm above a substrate holding the cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram depicting a non-contact cell manipulation system in accordance with some embodiments.

FIG. 2 depicts multiple views of components of the system of FIG. 1 in accordance with some embodiments.

FIG. 3 depicts an exploded view of the components of FIG. 2 in accordance with some embodiments.

FIG. 4 depicts multiple views of the components of FIG. 2 in accordance with some embodiments.

FIG. 5 depicts multiple views of the components of FIG. 2 in accordance with some embodiments.

FIG. 6 depicts multiple views of a non-contact multiphysics microfluidic probe in accordance with some embodiments.

FIGS. 7A-7B are images depicting example configurations of the tip of the probe of FIG. 6 in accordance with some embodiments.

FIG. 8 depicts multiple views of a probe holder in accordance with some embodiments.

FIG. 9 depicts multiple views of a probe adapter in accordance with some embodiments.

FIG. 10 is a flow chart depicting a non-contact cell sampling method in accordance with some embodiments.

FIG. 11 is a flow chart depicting a non-contact cell agent delivery method in accordance with some embodiments.

FIGS. 12A-12F are diagrams and plots depicting an example characterization of the system of FIG. 1 in accordance with some embodiments.

FIG. 13 is a diagram depicting an example application of the system of FIG. 1 in accordance with some embodiments.

FIG. 14 is a diagram depicting another example application of the system of FIG. 1 in accordance with some embodiments.

FIGS. 15A-15G are diagrams and plots depicting another example application of the system of FIG. 1 in accordance with some embodiments.

FIGS. 16A-16E are diagrams and plots depicting another example application of the system of FIG. 1 in accordance with some embodiments.

FIGS. 17A17-E are diagrams and plots depicting another example application of the system of FIG. 1 in accordance with some embodiments.

FIGS. 18A-18G are diagrams and plots depicting another example application of the system of FIG. 1 in accordance with some embodiments.

FIGS. 19A-19B are diagrams and images depicting another example application of the system of FIG. 1 in accordance with some embodiments.

FIGS. 20A-20E are plots showing example experimental details of the electric field of the system of FIG. 1 in accordance with some embodiments.

FIGS. 21A-21D are plots showing experimental details of the hydrodynamic flow confinement (HFC) of the system of FIG. 1 in accordance with some embodiments.

FIG. 22 is an image showing an example probe tip of the system of FIG. 1 in accordance with some embodiments.

FIGS. 23A-23D are plots showing example experimental results of the system of FIG. 1 in accordance with some embodiments.

FIGS. 24A-24C are plots and images showing example experimental characterization details of flat and spherical cells characterized by the system of FIG. 1 in accordance with some embodiments.

FIG. 25 is a diagram showing an example experimental scanning path of the system of FIG. 1 in accordance with some embodiments.

FIG. 26 shows example images of experimental multiple cell transfection by the system of FIG. 1 in accordance with some embodiments.

FIG. 27 shows example images and plots for experimental a multiple time point single cell biopsy using the system of FIG. 1 in accordance with some embodiments.

FIGS. 28A-28E show example experimental plots and images for excisional biopsy and real-time cytoplasm content monitoring experiments performed using the system of FIG. 1 in accordance with some embodiments.

FIGS. 29A-29C show example experimental images for an independent extraction of subcellular compartments experiment performed using the system of FIG. 1 in accordance with some embodiments.

FIG. 30 shows example experimental image results of surrounding cell regeneration after single-cell pick up utilizing the system of FIG. 1 in accordance with some embodiments.

FIG. 31 is a plot showing example experimental results of the system of FIG. 1 in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of non-contact cell manipulation. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a non-contact cell manipulation device, probe and method.

Disclosed is a multifunctional and multiphysics probe that can be precisely controlled to perform multiple manipulation and analysis procedures on living single-cells while in tissue-like culture. The multiphysics microfluidic probe (MMFP) includes fluidic apertures and electrodes which can be used to simultaneously confine reagents and electric signals to sub-cellular regions of single-cells. This work offers a multifunctional tool with unprecedented minimally invasive probing features for spatiotemporal single-cell analysis within tissue samples.

Further disclosed is a non-contact multiphysics microfluidic probe (MMFP) system with precise control to genetically manipulate and analyze living single cells. The probe is a multifunctional tool that combines the electropermealization (EP) technique with hydrodynamic flow confinement (HFC) to avoid physical contact with the cell. In certain instances the probe, or various probe components, can be fabricated utilizing 3D printing to enable rapid prototyping. In one embodiment the probe is manufactured via subtractive manufacturing means, such as milling the probe from a block of suitable material. The probe was developed by building on the chemical stimulation capabilities of the microfluidic multipoles concept. The evolving biological applications of microfluidic multipoles is based on using the microfluidic probe (MFP) technology to hydrodynamically confine a reagent to a target adherent cell cluster, without any physical contact. In one embodiment the disclosed system advances this concept by leveraging on previously developed 3D printing protocols to integrate 3D electrical components on the probe. In one embodiment the disclosed system utilizes standardized off the shelf electrical components attached to the probe. The vertical positioning of the probe, with respect to the cell, is also automated using image recognition models and validated structural mechanism correlations. In one embodiment, position of the probe with respect to the sample can be at an angle in the range of 10°-270°. These enabled precise simultaneous confinements of reagents and electric fields with a sub-cellular precision in order to facilitate multi-parametric manipulation operations while retaining cellular spatial configuration.

In one embodiment, combinatorial and sequential application of electropermealization, electroporation, electrolysis, electromanipulation, electrosensing, electroheating, hydrodynamic flow, electrical stimulation, chemical stimulation, and thermal confinement are used for single cell manipulation. The ability to probe the dynamics of single cells, while retaining spatial configurations, is also important for a better understanding of diseases, such as cancer. For example, crosstalk between immune cells in the tumor microenvironment have been reported to actively promote oncogenesis. Hence, studying tumor cells outside of their tissue environment does not give a full picture. For a fundamental understanding of these intra-tissue interactions, tools for manipulating and analyzing single cells while in their natural physiological environment are required. A few methods have now been successfully developed to probe single-cells while retaining their spatial configuration in tissue-like culture. However, these methods involve physical insertion of probes into the cell and can broadly be classified as nanopipettes and Fluid Force Microscopy (FluidFM). Some groundbreaking applications of these technologies include a combination of nanopipettes with dielectrophoresis (DEP) to achieve trapping of single molecules of DNA and protein, and a demonstration of the ability of cells to withstand several picoliters of cytoplasm extraction with the FluidFM. Nevertheless, since both nanopipettes and FluidFM involve physical penetration of the cell, they typically result in cell lacerations between 1-3% of the cell size which could be detrimental to subsequent cell function. Furthermore, passing fluids through nanoscale channels at velocities within physiological limits is challenging and requires complicated tooling, which makes these technologies impractical for multiplexed single-cell manipulation tasks.

The ability to extract subcellular contents from single cells without dissociating them from their physiological environment provide fundamental transcriptomic, proteomics and genomics insights while maintaining cellular and tissue integrity. Such capability is invaluable for increasing the fundamental knowledgebase on basic biology and organ developmental processes in tissues and living organisms, which is a field that still rely on data collected from multiple tissue sampling. Combining this feature with single-cell genetic manipulation facilitates sequential spatially resolved genetic manipulation, and overall omics sampling for multiplexed single-cells. This is indeed a groundbreaking addition to Human Cell Atlas mission, which is one of the most ambitious ongoing genomics projects with an aim of mapping all human cells.

Creating temporary pores on cell membrane via the application of electric pulses provides the basis for transporting molecules in and out of biological cells via EP. (Weaver et al., IEEE Trans plasma Sci 2000; 28: 24-33) This cell manipulation technique has been used extensively for the insertion of biomarkers, proteins, drugs, genes and other macromolecules (e.g., Ca2+ ions) into cells. (Jordan et al., Electroporation and electrofusion in cell biology. Springer Science & Business Media, 2013) In general, these experimental outcomes have been in agreement with the available theoretical framework. (Weaver et al., Plant cell electroporation and electrofusion protocols. Springer, 1995, pp 3-28) (Weaver et al., Bioelectrochemistry Bioenerg 1996; 41: 135-160) However, a majority of these studies have been carried on suspended cells and preliminary indications suggest that adaptation of these theories will have to be made for applications on cells in adherent cultures. (Jordan et al., Methods 2004; 33: 136-143) Hence, more comprehensive characterizations are required for better understanding of the EP-based transfer of molecules into adherent cells.

The probe can also be used to extract portions of the cell's cytoplasmic and nucleoplasmic contents via incisional cell biopsy or completely lyse the cell to collect all of its cytoplasmic and nucleoplasmic contents via excisional cell biopsy dependent on the electric field characteristics applied to the probe's electrode. Furthermore, combination of the described biopsies with several other force fields, such as microfluidic flow, electric, acoustic and magnetic, can be also used to sequentially extract the cells cytoplasmic and nucleus contents. Additionally, whole single cells can be retrieved from tissue samples by using the probe to simultaneously expose the target-cell to an enzymatic solution, such as trypsin, and an optimized dissociation temperature via isolated joule heating through the electrodes. Moreover, parts from single cell membranes can be retrieved from tissue samples by using the probe to electrolyse cells and hydrodynamically collect cell membrane components, and/or by using electro-generated forces such as dielectrophoresis. Additionally, cell nucleus and nuclear membrane can be targeted and collected by optimizing the procedures after electrolyzing and/or chemically-lysing the cell membrane.

In another application the probe design can be modified to perform continuous multiple cell sampling, or sub-cellular content sampling, with oil droplet compartmentalization or other droplet microfluidic systems. In one embodiment, the system further comprises an integrated microfluidic droplet generator to separate the extracted contents of multiple cells with oil plugs. In one embodiment, the system further comprises integrated fluidic networks, resistors, channels, incubation chambers, filters, valves, and/or reservoirs. In one embodiment, the system further comprises electrodes integrated within the probe's 3D body for electro-sensing, electro-heating, electrophoresis, electro-osmosis, electro-pumping, electro-wetting. In one embodiment, the system further comprises electrodes on the probe's surface for micro-positioning control based on capacitance measurement.

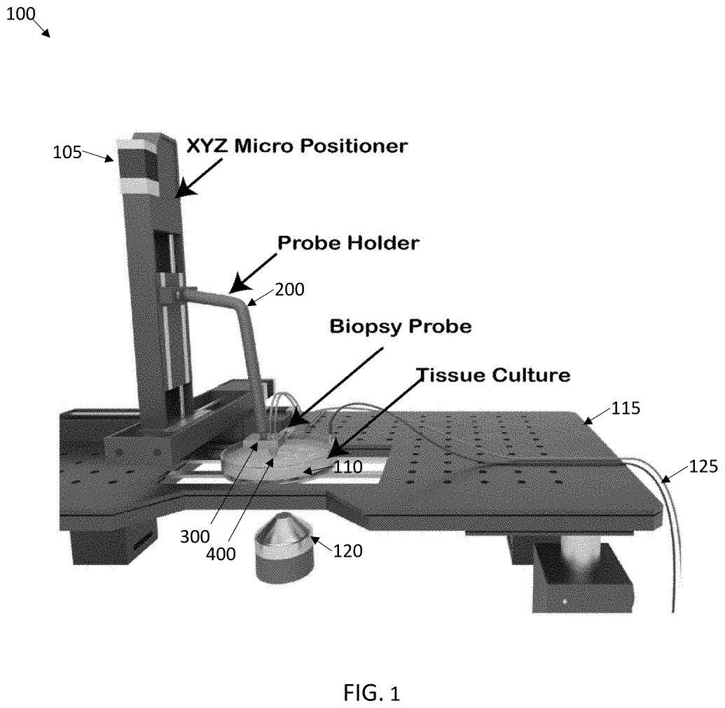

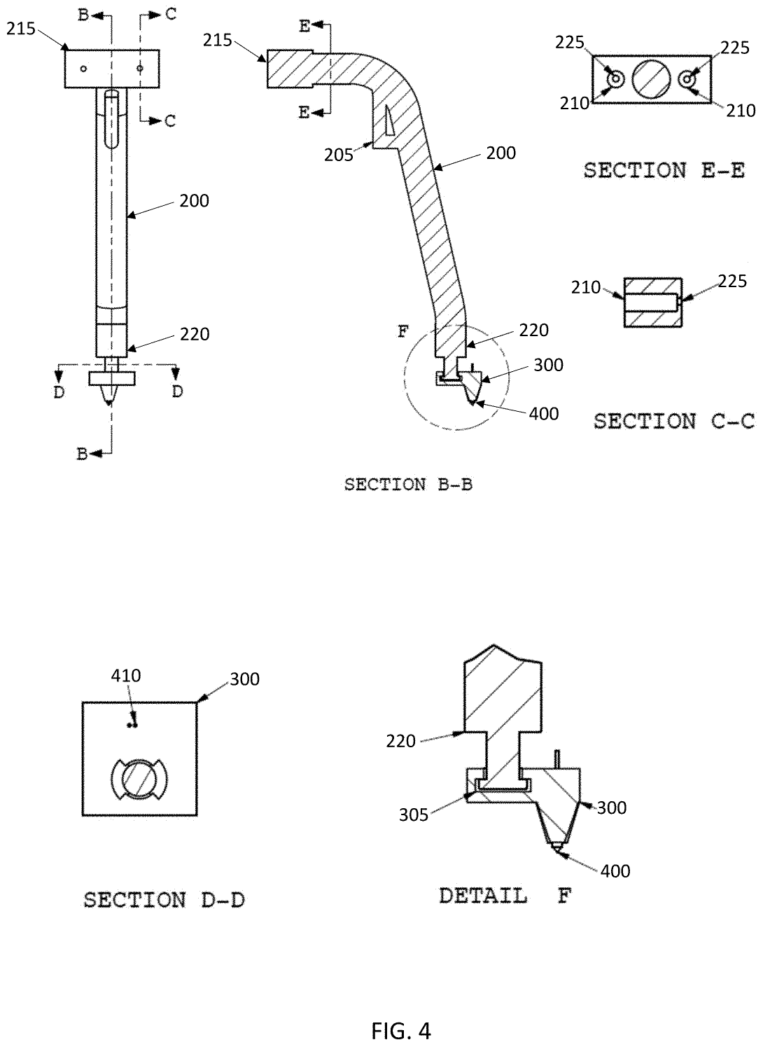

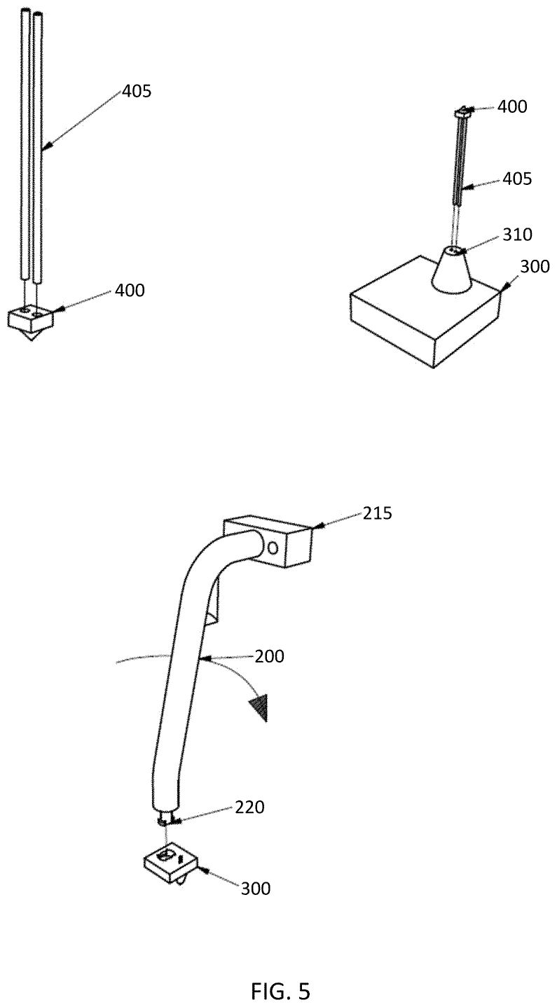

FIGS. 1 through 9 depict an example embodiment of a system 100 and a multiphysics microfluidic probe 400 configured to perform non-contact cell manipulation utilizing electropermealization in combination with hydrodynamic flow confinement. FIG. 1 shows one embodiment of the non-contact cell manipulation system 100. The system includes a supporting member 115, a three-dimensional micro-positioner 105, a probe holder 200, a probe adapter 300 and a non-contact multiphysics microfluidic probe 400.

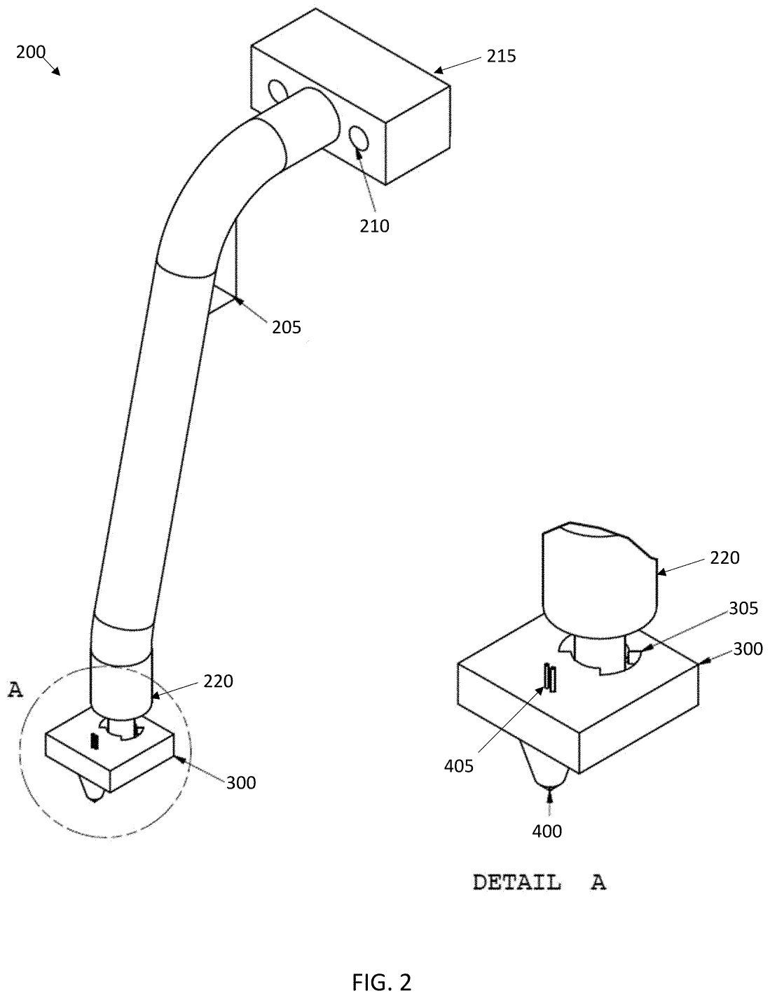

The probe holder 200 has a proximal end 215 and a distal end 220. The proximal end 215 is connected to the micro-positioner 105. Further details of the probe holder are shown in FIGS. 2, 3, 4, 5, and 8. In one embodiment, the probe holder can include other six degrees of freedom robotic or manual manipulation components with rotational envelops.

In one embodiment the probe adapter 300 is removably connected to the distal end 220 of the probe holder 200. In one embodiment the probe adapter is permanently fixed to the distal end 220 of the probe holder 200. Further views and details of the probe adapter are shown in FIGS. 2, 3, 4, 5, and 9 and described below. In one embodiment, the probe adapter 300 can accommodate a plurality of probes 400, whereby based on the biological target a specific probe 300 can be made to engage with the sample.

The probe 400 is fluidly and electrically connected to the probe adapter 300. In one embodiment, the probe 400 includes at least one electrode 445 and at least one aperture 455. In one embodiment, the probe 400 includes at least one electrode 445 and at least one outlet aperture 455. In one embodiment, the probe 400 includes at least one electrode 445 and at least on inlet aperture 450. In one embodiment, the probe 400 includes at least one electrode 445, at least one inlet aperture 450, and at least one outlet aperture 455. In one embodiment, probe 400 is configured to utilize one or more of electropermealization, electrolyzing, electroportation, electrosensing, electroheating, electrical stimulation, electromanipulation, hydrodynamic flow confinement and thermal flow confinement. For example, in one embodiment, probe 400 is configured to utilize electropermealization and electromanipulation in combination with hydrodynamic and thermal flow confinement to perform non-contact cell manipulation.

In one embodiment the probe adapter 300 is removably connected to the probe holder 200 via a twists and lock connection. In one embodiment the probe adapter 300 is removably connected to the probe holder 200 via a fastener. In one embodiment the probe adapter 300 is permanently connected to the probe holder by design, or via a weld, an adhesive, or other permanent boding technique. In one embodiment, the probe 400 is a 3D printed part.

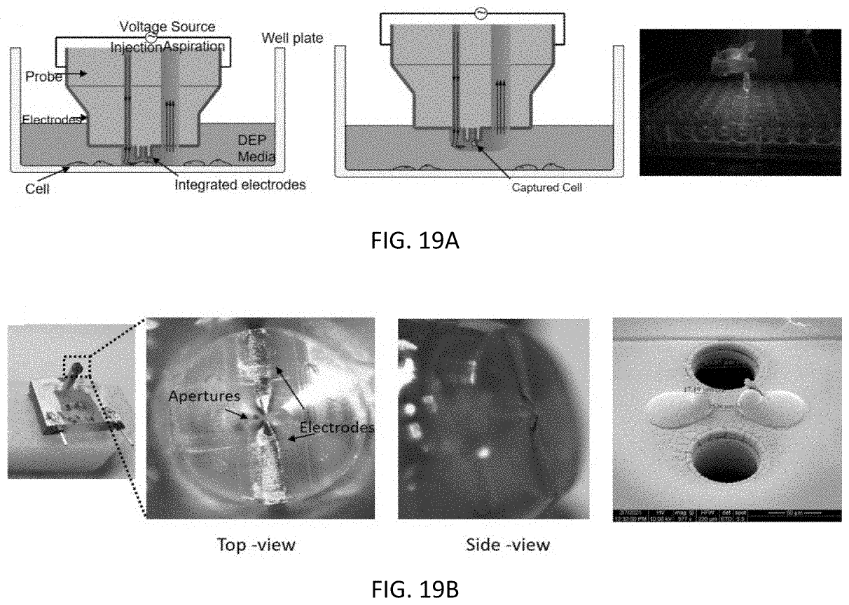

The system further includes electrical conduit 125 to supply power to the electrode 445, a system for probe calibration and positioning 120, and a cell culture substrate 110. In one embodiment the cell culture substrate 110 is coated in a conductive substrate material, such as indium tin oxide (ITO) for example. In one embodiment the electrode 445 and the coated substrate material of the substrate 110 form a pin-plate electrode system. In another embodiment, the cell culture substrate is non-conductive, and two or more electrodes are located on the probe 400, as shown in FIGS. 19A-19B and described below. In one embodiment, a subset of the at least one electrode is configured as a counter-electrode. In one embodiment, the cell culture is a thin tissue biopsy slice, an artificial 3D cell culture, and a native in vivo tissue.

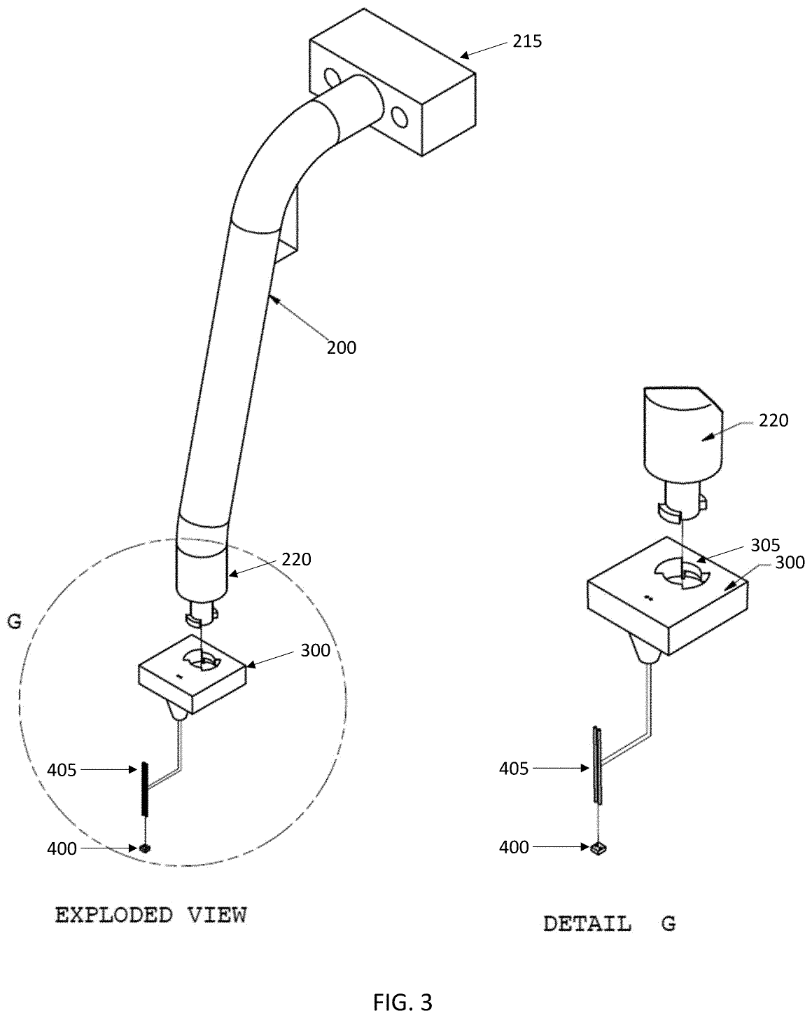

FIGS. 2, 4, and 5 shows further details of the connection of the probe holder 200, the adaptor 300, and the probe 400. FIG. 3 shows an exploded view of the components in FIG. 2. The probe holder can have a cylindrically shaped stem 230 with multiple bends. In one embodiment an angle bar support 205 is incorporated into the probe holder 200. The proximal end 215 of the probe holder 200 includes countersinks 210 and through holes 225 configured to accept fasteners to attach the probe holder 200 to the micro-positioner 105. In one embodiment the countersinks 210 are 5 to 10 mm diameter and 1 to 3 mm deep. In one embodiment the through holes 225 are 1 to 5 mm in diameter.

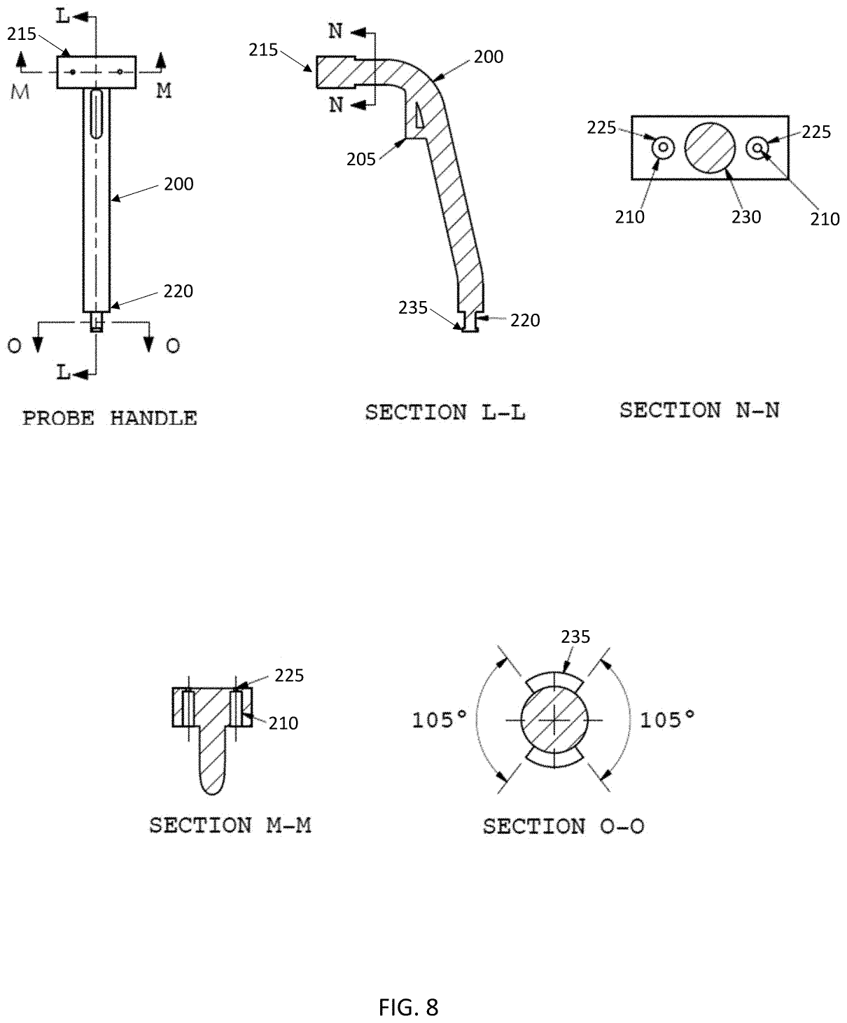

In one embodiment, the adapter 300 removably connects via a twist and lock motion to the distal end 220 of the probe holder 200. The distal end 220 includes a male twist and lock connector. Details of the geometry of the male twist and lock connector are shown in Section O-O of FIG. 8. The connector includes a pair of lock lips 235 with about 105° arc separation between one another.

The probe 400 connects to pins 405 via a friction fit with apertures 410. The pins 405 are hollow cylindrical tubes that are configured to act as inlet and outlet fluid conduits for the apertures 450, 455. In one embodiment, the pins 405 provide a friction fit to connect the probe 400 to the adapter 300. In one embodiment, the pins 405 are glued to, or otherwise adhered to, the probe 400.

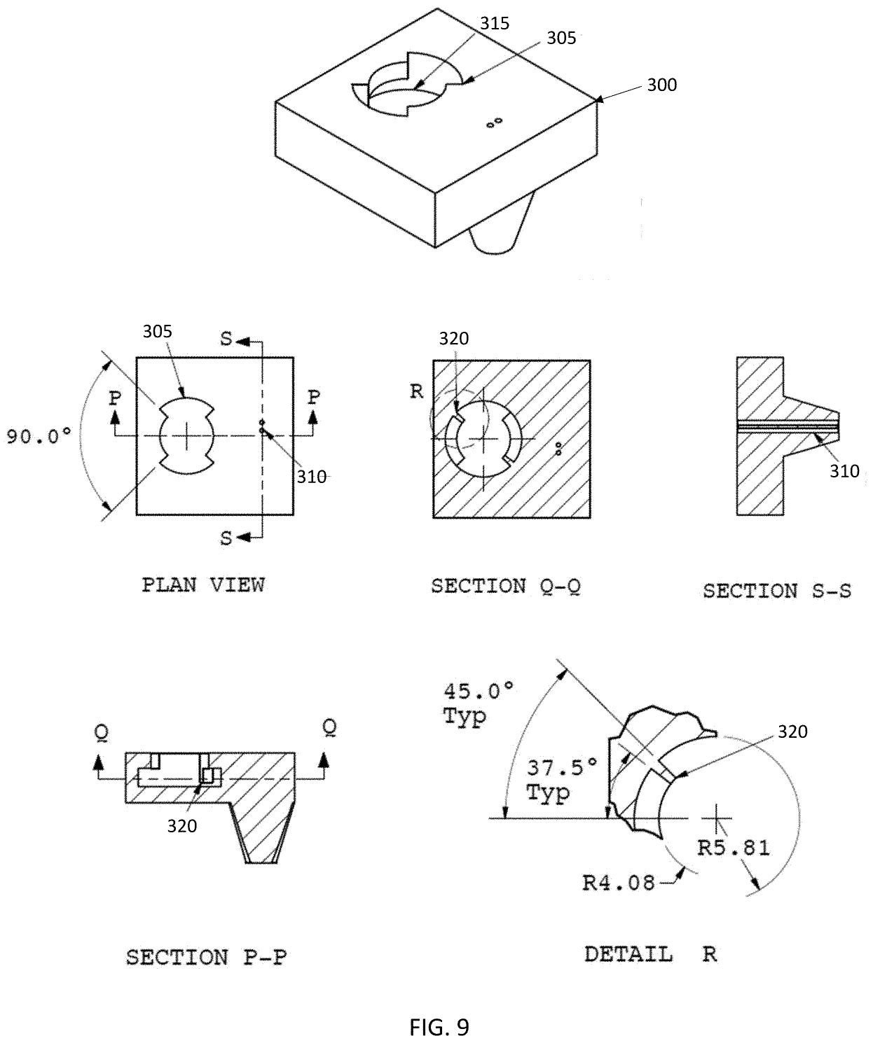

An example configuration of the adapter 300 is shown in FIG. 9. An isometric view along with a plan view, sectional views and details views are shown. The adapter 300 includes a cuboid top section and a conical bottom section protruding from one face of the cuboid. A female twist and lock grove 305 is located on the cuboid face opposite the conical section. In one embodiment, the depth 315 of the lock grove 305 is 3 to 6 mm. Through holes 310, configured to receive the pins 405, extend from the cuboid face including the lock grove 305 to the opposite cuboid face where the conical section is located, and proceed to extend through the conical section. In one embodiment the through holes 310 are 0.1 to 1.5 mm in diameter. Stoppers 320 are positioned on the underside of the twist and lock groove 305 lips and are configured to limit the rotation of the adapter 300 when connection of the adapter 300 to the probe holder 200 is performed. The twist and lock grove 305 lips extend through an arc of 80 to 100 degrees and the stoppers 320 extend through an arc of 5 to 10 degrees.

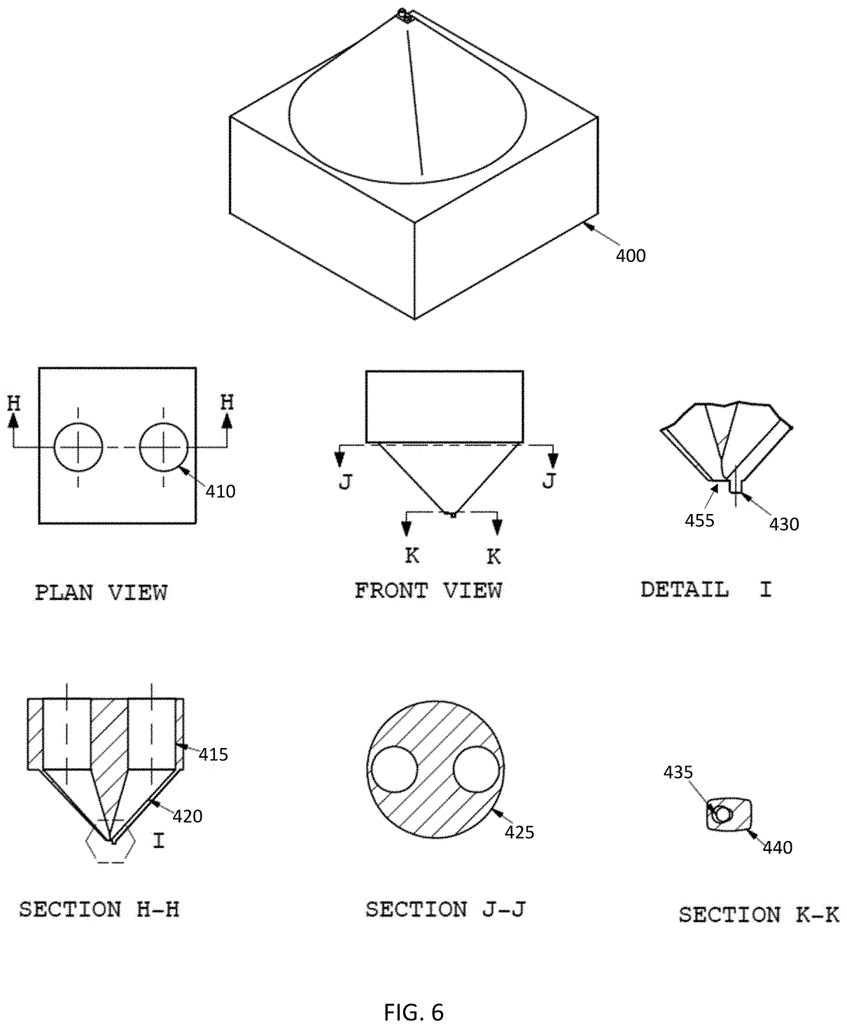

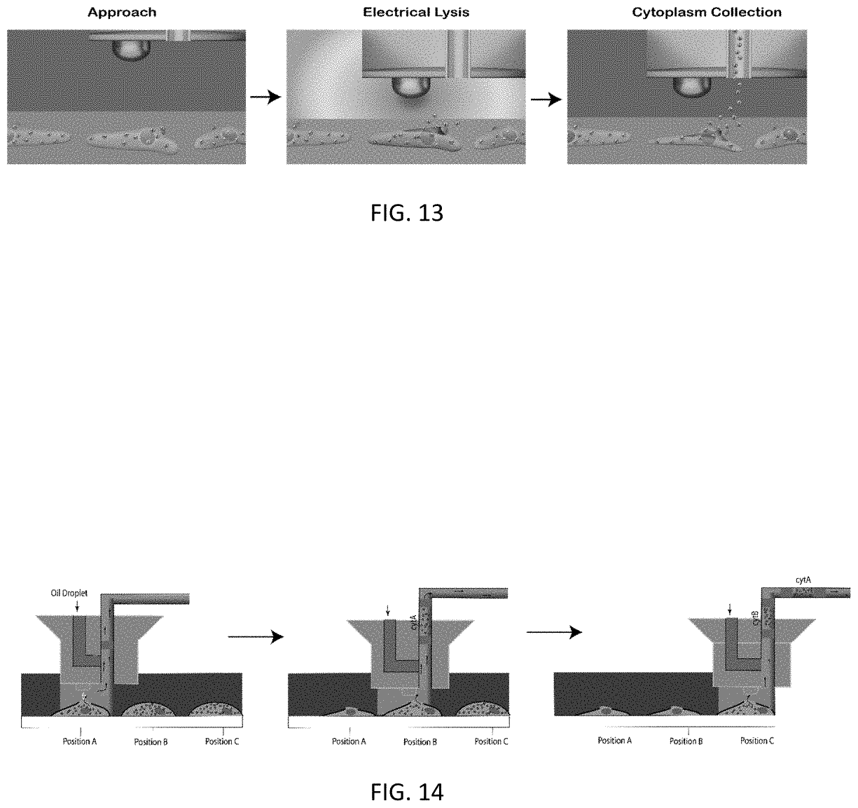

FIG. 6 shows multiple views of one embodiment of the probe 400, where the probe 400 is a single aperture probe. An isometric view along with a plan view, front view, detail view, and sectional views are shown. The probe includes a cuboid top section and a conical bottom section protruding from one face of the cuboid. The tip of the conical section is flat and forms the tip of the probe 400. Apertures 410 are position on the face of the cuboid opposite the conical section. The apertures 410 are connected to through holes 415 extending through the cuboid section, where the through holes 415 are further connected to converging holes 420 that extend to the tip of the conical section and form a single aperture 455. In one embodiment the apertures 410 are 1 μm to 1.5 mm in diameter. The top section 425 of the conical section is shown in Section J-J. While a conical section is shown, other geometries with a converging tip can also be utilized. The angle of the convergence is 30 to 80 degrees, and the height of the convergent tip is 10 μm to 5 mm. Detail I shows an end cylindrical boss 430 present in one embodiment. In one embodiment this boss 430 is 10 to 100 μm in diameter and 10 to 100 μm in height. The base section 440 of the conical section is shown in Section K-K where an electrode 435 is positioned. FIGS. 13 and 14 show additional example embodiments of single aperture probes 400. In one embodiment, the probe 400 is configured to perform compartmentalized multiple cell extraction.

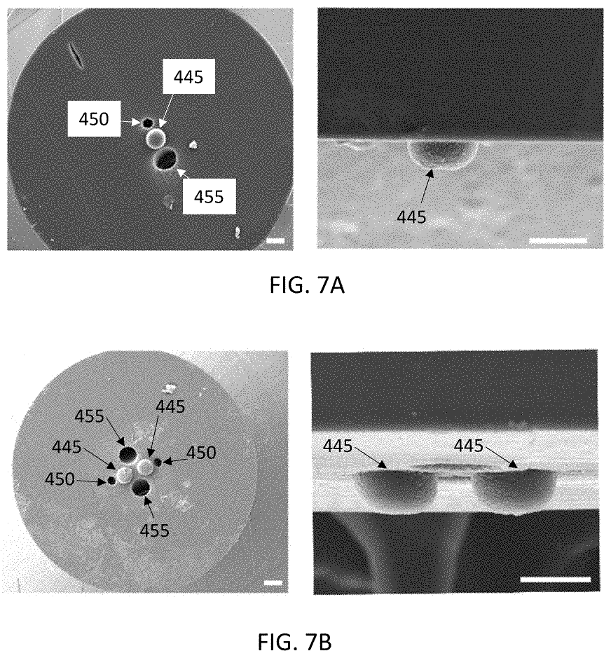

FIGS. 7A-7B show images of example tip configurations of embodiments of the probe 400 that include multiple apertures. The probe 400 comprises at least one inlet aperture 450, as least one outlet aperture 455, and at least one electrode 445. In one embodiment the electrode 445 is positioned between the apertures (450, 455). In one embodiment electrode 445 and the apertures (450, 455) are configured to perform electropermealization and electromanipulation in combination with hydrodynamic flow and thermal flow confinement. In one embodiment the electrode 445 has a radius of 0.1 to 100 μm and a height of 0.1 to 1000 μm. In one embodiment the electrode 445 has a radius of about 25 μm and a height of about 25 μm. In one embodiment the electrode 445 is a dome shape. In one embodiment the electrode 445 is a conical shape. In one embodiment the electrode 445 is a spiked shape. In one embodiment the electrode 445 is a cylindrical shape. In one embodiment the electrode is a pin shape. In one embodiment the electrode is an arc shape. In one embodiment the electrode is a spike shape. In one embodiment the electrode is a suspended shape. In one embodiment the electrode is a triangular shape. In one embodiment the electrode is a pyramidal shape. In one embodiment the electrode is integrated with nanotubes. In one embodiment the inlet aperture 450 has a radius of 0.5 to 500 μm, and the outlet aperture 455 has a radius of 0.5 to 500 μm. In one embodiment the inlet aperture 450 has a radius of about 15 μm, and the outlet aperture 455 has a radius of about 30 μm. In one embodiment the inlet aperture 450 and outlet aperture 455 are spaced 1 to 2000 μm center-to-center apart. In one embodiment the inlet aperture 450 and outlet aperture 455 are spaced about 100 μm center-to-center apart. In one embodiment, the electrode 445 is positioned centrally between the inlet aperture 450 and the outlet aperture 455.

As shown in FIGS. 7A-7B, various arrangements and numbers of the electrode(s) and apertures are possible. The configurations shown in the figure are merely examples, and other configurations are possible. FIGS. 7A-7B show scanning electron microscopy micrographs with the top (left) and side (right) views of the two apertures/one electrode (FIG. 7A) and four apertures/two electrodes (FIG. 7B) probe 400. The scale bars are 50 μm. FIG. 7A shows the two aperture/one electrode configuration comprising one inlet aperture 450, one outlet aperture 455, and one electrode 445. In this embodiment, the electrode 445 is centrally located on the tip of the probe 400, and centrally located between the inlet aperture 450 and outlet aperture 455. Additionally, the electrode 445 protrudes from the tip of the probe 400.

FIG. 7B shows the four aperture/two electrode configuration comprising two inlet apertures 450, two outlet apertures 455, and two electrodes 445. In this embodiment, the two electrodes 445 are positioned linearly about the center of the tip of the probe 400 and are spaced 75 μm center to center. In one embodiment the electrodes 445 are spaced 0.1 μm or more apart center to center. The two inlet apertures 450 are positioned in the same linear direction as the electrodes 445, with the electrodes 445 being positioned centrally between the inlet apertures 450. The outlet apertures 455 are positioned linearly with one another and are positioned perpendicular to the linear direction established by the electrodes 455. Additionally, the electrodes 445 are positioned centrally between the outlet apertures 455. These positions and qarrangements are merely examples, and any suitable multipolar fluidic and electric configurations are possible.

In one embodiment, the probe 400 is made from non-conductive materials, such as polymers, and are coated with a conductive layer. In one embodiment, the probe 400 is made from a conductive material. In one embodiment, the probe is coated with a patterned conductive layer covering the electrode 445. In one embodiment, the probe is coated with a patterned conductive layer cover to create two or more electrodes 445, thus leading to a configuration where the two or more electrodes 445 can be utilized as counter-electrodes and no conductive substrate 110 is necessary.

In one embodiment, the probe is coated with a conductive substrate. In one embodiment, the probe is coated with a gold substrate. In one embodiment, the gold substrate is patterned to form isolated and independently addressable electrodes. In one embodiment, the bottom substrate is a regular cell culture plate. In one embodiment, the bottom substrate is a tissue slice holder. In one embodiment, the bottom substrate is the target 3D tissue itself. In one embodiment, the system further comprises a conductive cell culture substrate. In one embodiment, the conductive cell culture substrate comprises a glass slide coated in an ITO substrate. In one embodiment, the electrode and the ITO coated substrate form a pin-plate electrode.

In one embodiment sampling with compartmentalization is performed. The probe design is modified to perform an oil droplet compartmentalized sequential collection or other droplet microfluidic mechanism. In the example shown in FIG. 14, the inlet aperture 450 is connected inside the probe 400 directly an outlet flow conduit above the outlet aperture 455. When sampling is performed, oil droplets are provided by the inlet aperture 450 to compartmentalize the sampling. In one embodiment, a plurality of inlet apertures 450 and outlet apertures 455 are configured to perform sampling with compartmentalization. These positions and arrangements are only examples, and any other suitable multipolar fluidic and electric configuration is possible. In one embodiment the inlet aperture 450 and outlet aperture 455 are 0.1 to 1000 μm in diameter. In one embodiment the flow rate is 0.001 to 50 μL/min.

In one embodiment, the system 100 and probe 400 are configured for in vivo applications. In this embodiment the probe is positioned directly above target cells while within tissue samples in vivo. Two or more electrodes 445 that are not electrically connected to one another are utilized. The two or more electrodes 445 can be made from a patterned conductive coating, via additional manufacturing of conductive electrodes, and/or by using sacrificial layers with the probe design.

In one embodiment the system 100 includes an imaging system. In one embodiment the imaging system is also the system used for calibration and positioning 120. In one embodiment the imaging system is a microscope. In one embodiment, the imaging system is a high magnification camera. In one embodiment, the imaging system comprises a single unit objective lens. In one embodiment, the imaging system comprises an optical fiber. In one embodiment the imaging system is positioned beneath the substrate 110 as a visual aid. In one embodiment the imaging system is positioned on the side of the substrate 110. In one embodiment, the imaging system is positioned above the substrate 110. In one embodiment a combination of different imaging systems at a plurality of positions are utilized.

In some embodiments, the devices of the present invention may operate in conjunction with a computer platform system, such as a local or remote executable software platform, or as a hosted internet or network program or portal. In certain embodiments, portions of the system may be computer operated, or in other embodiments, the entire system may be computer operated. As contemplated herein, any computing device as would be understood by those skilled in the art may be used with the system, including desktop or mobile devices, laptops, desktops, tablets, smartphones or other wireless digital/cellular phones, televisions or other thin client devices as would be understood by those skilled in the art.

The computer operable component(s) may reside entirely on a single computing device or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data formats between two or more computing devices. There are no limitations to the number, type or connectivity of the databases utilized by the system of the present invention. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

The present invention also provides a method of non-contact cell manipulation. For example, in certain embodiments, the method comprises the use of the system and probe, as described herein, to sample the contents of individual cells within a cell population. For example, the method can be used to isolate any or all of the intracellular components of a cell, including but not limited to, proteins, nucleic acids, organelles, nuclear components, metabolites, and the like.

In one aspect, the present invention also proves a method of non-contact delivery of an agent of interest to an individual cell within a cell population. For example, the method can be used to deliver a nucleic acid molecule, protein, peptide, small molecule, dyes, nanoparticles, quantum dots, or the like to the intracellular compartment of a cell. For example, the method can be used to deliver special reagents inside selected cells within tissues, for in situ biosensing, in situ hybridization, in situ reactions, in situ chain reactions, and/or in situ polymerase chain reaction.

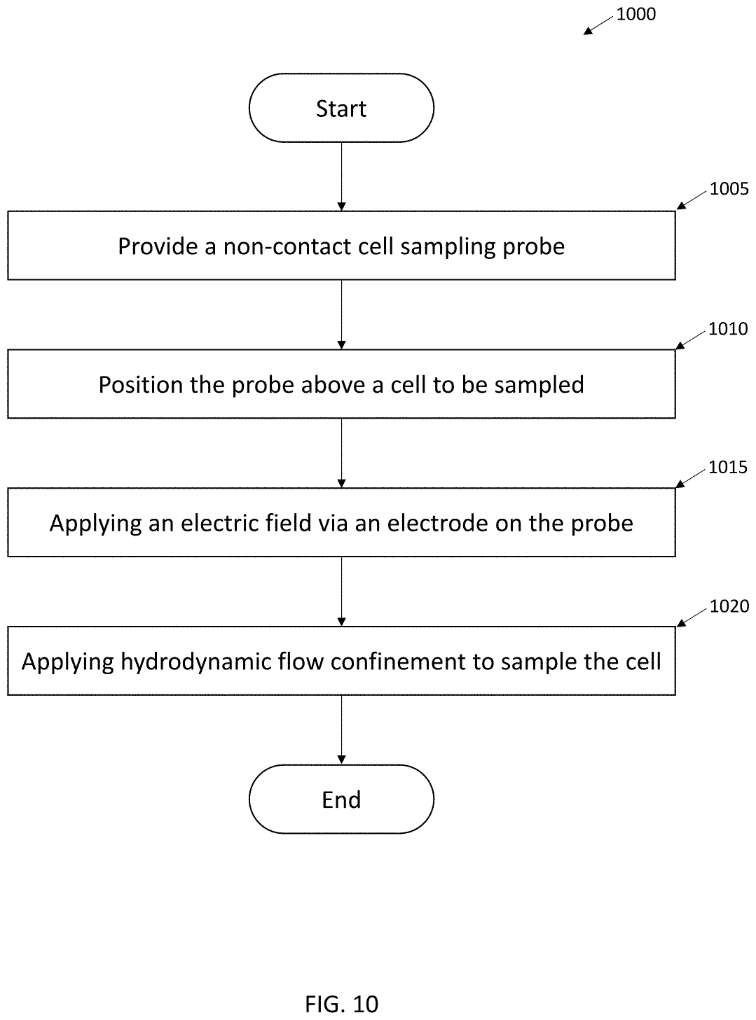

FIG. 10 depicts a non-contact cell sampling method 1000. The method 1000 starts at Operation 1005, where a non-contact manipulation probe 400 is provided. An example of this probe can be probe 400 of system 100. In one embodiment, the probe 400 includes at least one aperture 455 and at least one electrode 455. In one embodiment, the probe 400 includes at least one inlet aperture 450, at least one outlet aperture 455, and at least one electrode 445 positioned between the apertures. The probe 400 is configured to perform cell manipulation using electropermealization in combination with hydrodynamic flow confinement.

At Operation 1010 the probe is positioned above a cell to be manipulated and sampled. The positioning can be controlled via a three-dimensional micro-positioner 105 of the system 100. Additionally, the vertical position above the cell can be initially established via a zeroing calibration method based on the horizontal displacement when the probe 400 contacts a substrate 110. In one embodiment, the calibration is done by visual inspection. In one embodiment, the calibration is done via digitized image recognition algorithms. In one embodiment, the positioning is manual controlled. In one embodiment, the positioning is digitally controlled. In one embodiment, the positioning is automatically controlled via digital image recognition and related algorithms. In one embodiment, the positioning system may integrate optical components, such as optical fibers, or miniaturized sensors, such as force or displacement transducers, for position control. In one embodiment, the positioning system integrates electrical components, such as proximity sensors, capacitive censors, impedance sensors, for position control. In one embodiment the probe is positioned about 20 μm above the substrate 110 holding the cell sample.

At Operation 1015 an electric field is applied to the cell to be sampled via the electrode 445 on the probe 400. In one embodiment the electric field is pulsed. The electrode operation parameters can include an operating voltage of 0 to 150 V, a 0 to 500 MHz AC or DC current, and a duration from 0-150 minutes. Example electric field parameters for various sampling examples are described below in the Experimental Examples section.

In certain embodiments, the parameters are varied depending on the application desired. For example, in one embodiment, the method comprises sampling of only a portion of the cytoplasmic content of the cell. In one embodiment, the method comprises sampling of the entirety of the cytoplasmic content of the cell. The field strength can vary depending on the cell type sampled. In one embodiment, for partial sampling the electric field strength is 0 to 1000 V/cm, and for complete sampling the electric field strength is greater than 1000 V/cm. In one embodiment, for partial sampling the electric field strength is 0 to 1500 V/cm, and for complete sampling the electric field strength is greater than 1500 V/cm. In one embodiment, for partial sampling the electric field strength is 0 to 2000 V/cm, and for complete sampling the electric field strength is greater than 2000 V/cm. In one embodiment, for partial sampling the electric field strength is 0 to 2500 V/cm, and for complete sampling the electric field strength is greater than 2500 V/cm. In one embodiment, for partial sampling the electric field strength is 0 to 3000 V/cm, and for complete sampling the electric field strength is greater than 3000 V/cm.

In one embodiment, the method comprises whole cell-sampling without disruption of the cell membrane, which allows for sampling of the nucleus and nucleic acid molecules and sequences contained therein. In one embodiment, the method comprises the use of the probe to administer an enzymatic solution to the cell to disrupt the cell adherence in culture. In one embodiment, the enzymatic solution comprises trypsin. In one embodiment, the enzymatic solution comprises TrypLe. In one embodiment the enzymatic solution concentration is 0.25 to 1.25%. In one embodiment, the method comprises using the probe to provide localized heating of the cell microenvironment to aid in the enzymatic disruption of the cell. In one embodiment, after complete sampling of the cell's cytoplasm, electrical lysis of the cytoplasm is combined with additional force fields to retrieve the cell's nucleus.

At Operation 1020 hydrodynamic flow confinement is applied via the apertures 450, 455 to perform manipulation of the cell, in this case sampling the cell. Fluid is supplied via the inlet aperture 450 and retrieved via the outlet aperture 455. The fluid flow operational parameters can include a flow rate of 0 to 100 μL/min, and a duration from 0 to 300 minutes for some applications or continuous flow for other applications. Example fluid flow parameters for various sampling examples are described below in the Experimental Examples section.

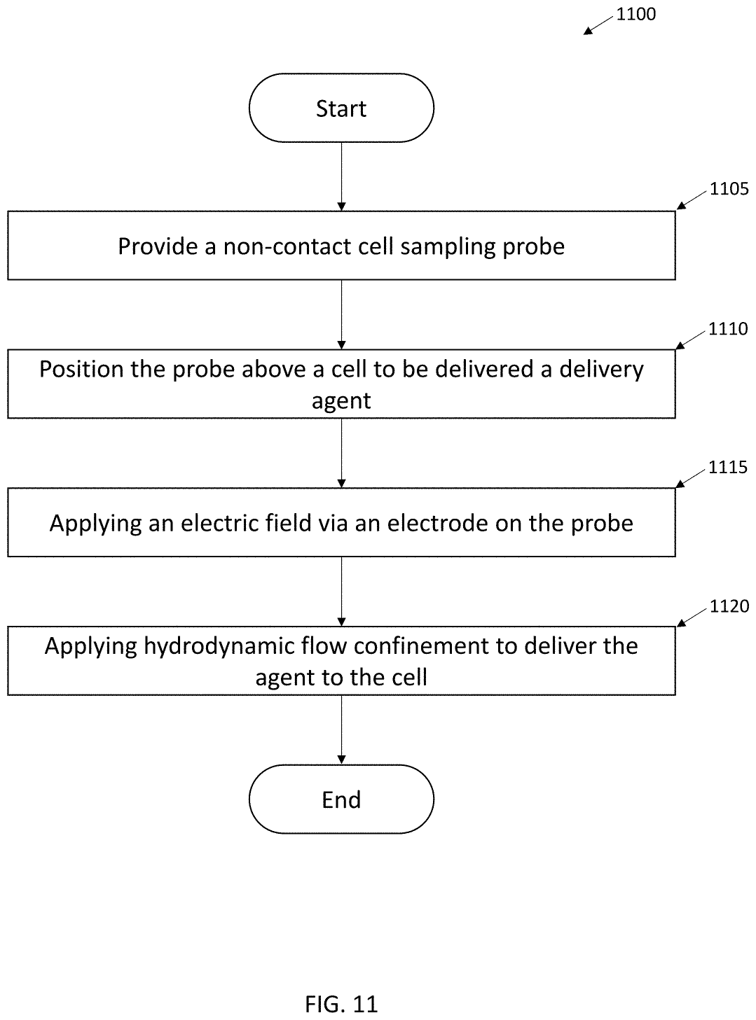

FIG. 11 depicts a non-contact cell agent delivery method 1100. The method 1100 starts at Operation 1105 where a non-contact manipulation probe 400 is provided. An example of this probe can be probe 400 of system 100. In one embodiment, the probe includes at least one aperture 455 and at least one electrode 445. In one embodiment, the probe 400 includes at least one inlet aperture 450, at least one outlet aperture 455, and at least one electrode 445 positioned between the apertures. The probe 400 is configured to perform cell manipulation using electropermealization in combination with hydrodynamic flow confinement.

At Operation 1110 the probe is positioned above a cell to be manipulated and delivered the agent. The positioning can be controlled via a three-dimensional micro-positioner 105 of the system 100. Additionally, the vertical position above the cell can be initially established via a zeroing calibration method based on the horizontal displacement when the probe 400 contacts a substrate 110. In one embodiment, the calibration is done by visual inspection. In one embodiment, the calibration is done via digitized image recognition algorithms. In one embodiment, the positioning is manual controlled. In one embodiment, the positioning is digitally controlled. In one embodiment, the positioning is automatically controlled via digital image recognition and related algorithms. In one embodiment the probe is positioned about 20 μm above the substrate 110 holding the cell sample.

At Operation 1015 an electric field is applied to the cell to be delivered the agent via the electrode 445 on the probe 400. In one embodiment the electric field is pulsed. The electrode operation parameters can include an operating voltage of 0 to 150 V, a 0-500 MHz AC or DC current, and a duration from 0-15 minutes. In one embodiment the current is pulsed. Example electric field parameters for various agent delivery examples are described below in the Experimental Examples section.

At Operation 1120 hydrodynamic flow confinement is applied via the apertures 450, 455 to perform manipulation of the cell, in this case delivering the agent to the cell. Fluid is supplied via the inlet aperture 450 and retrieved via the outlet aperture 455. The fluid flow operational parameters can include a flow rate of 0 to 100 μL/min, and a duration from 0-300 minutes for some applications or continuous flow for other applications. Example fluid flow parameters for various agent delivery examples are described below in the Experimental Examples section.

In one embodiment the manipulation is performed on a cell still within its original tissue organization. In one embodiment the manipulation performed is a single-cell biopsy operation. In one embodiment the manipulation performed is a heat assisted single-cell tweezer operation. In one embodiment, the manipulation performed is a single-cell electromanipulation operation. In one embodiment, the manipulation performed is a combination of electropermealization, electromanipulation, and heat assisted single-cell tweezer operation.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

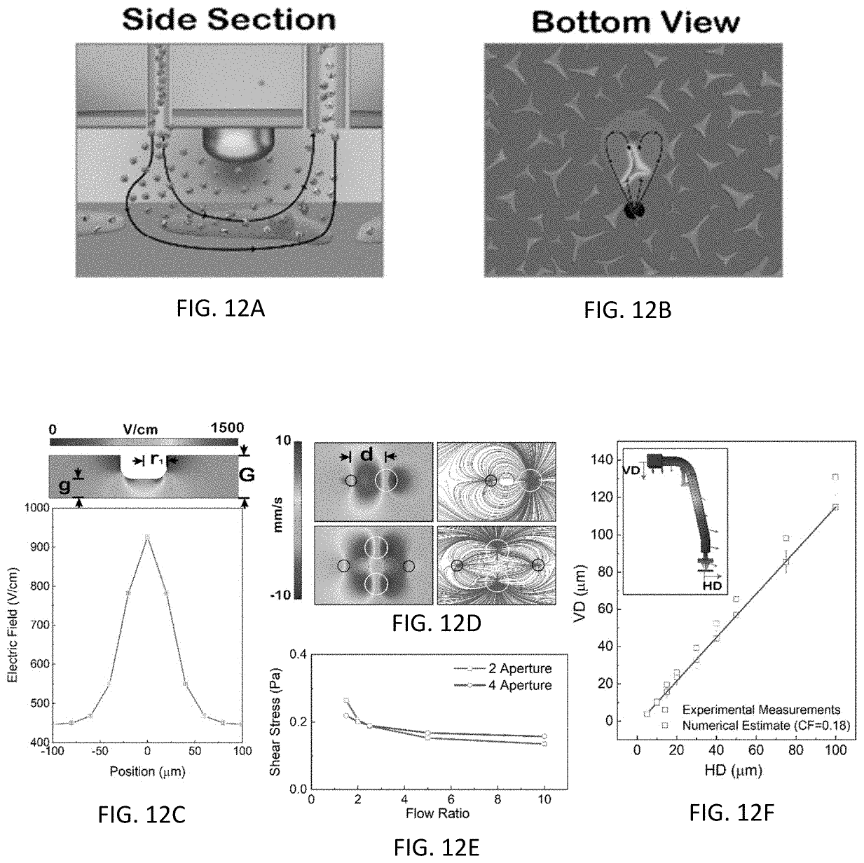

FIGS. 12A-12F show an experimental characterization of an example cell manipulation system 100, while FIGS. 13, 14, 15A-15G, 16A-16E, 17A-17E, and 18A-18G show example experimental applications of the system 100.

This work demonstrates the development and fabrication of the probe 400 as a non-contact single-cell manipulation and analysis tool, with multiple spatiotemporal resolved applications. The probe 400 performs a wide range of multiphysics enabled manipulation tasks while retaining cell viability and overall spatial arrangement. The increased functionality provided by this multiphysics probe 400 provides time dependent development studies on single-cells in tissue-like cultures. Incidentally, it also yielded new insights into the dependence of cell morphology on the EP of cells in tissue-like cultures, which is relevant to the electro-chemotherapy field. Given the versatility of the probe 400, there are endless possible applications for single cell analysis.

The probes 400 described herein were fabricated by 2-photon lithography-based 3D printing and their surfaces were made electrically conductive by direct gold sputtering. (Anscombe, Nat Photonics 2010; 4: 22-23) The hump shaped electrode 445, which provides a concentrated electric field, was located on the tip of the probe 400 and the counter electrode comprised of a transparent conductive ITO coated glass substrate. In the two-aperture (microfluidic dipole) and single hump (monoplex) electrode configuration adapted for single-cell targets, the size of the electrode 445 was 25 μm base radius and height, whereas the fluid apertures were designed with radius' of 15 μm and 30 μm for the inlet and outlet apertures respectively 450, 455. (Safavieh et al., Sci Rep 2015; 5: 11943) A 100 μm center-to-center spacing (S) between both apertures (450, 455) allowed for an electrode 445 placement at the central position. These dimensions define the fluidic confinement and electric field footprints. To achieve multiplexed probing of single-cells, the electrodes 445 and apertures (450, 455) were doubled to a four-aperture (microfluidic quadrupole) and two hump electrode (duplex) configuration respectively. (Qasaimeh et al., Nat Commun 2011; 2: 464)

During operation, the device works in a pin-plate electrode setup, with the hump representing the “pin” electrode 445 and an ITO coated substrate as the counter “plate” electrode. The hump electrode 445 confines an electric field to the target cell and a push-pull flow configuration through the apertures creates a hydrodynamic flow confinement (HFC) of the reagent around the cell. (Juncker et al., Nat Mater 2005; 4: 622-627) Using the spherical coordinate system, the electric field (E) between the hump and the substrate is given by Equation 1, where V is the applied voltage, r₁ is the electrode's radius, r₂ is the radial transform of the electrode-substrate gap, and r is the curvature radius of interest. (Pohl et al., Biophys J 1971; 11: 711-727) The calculated electric field experienced across the substrate of the “hump-plate” setup shows sharpening to display confinement to the target area, see FIG. 12C.

$$E = V \frac{{r}_{1} {r}_{2}}{{r}^{2} ( {r}_{2} - {r}_{1} )} \qquad ( 1 )$$

For a given tip-substrate spacing (G), the shape and span of the HFC is dependent on the ratio between the outlet and inlet flow rates (Qₒᵤₜ/Qᵢₙ) while, the magnitude of the flow velocity is dependent on the flow rates at the inlet and outlet apertures 450, 455, see FIG. 12D. (Goyette et al., Nat Commun 2019; 10: 1781) Using a point-source aperture approximation, the exact solution of the microfluidic dipole's velocity profile is defined by Equation 2, where x and y are the positional coordinates outlining the HFC footprint under the Cartesian coordinates, d is the spacing between the apertures, G is the tip-substrate gap, and α is (Qₒᵤₜ/Qᵢₙ).

$$\vec{v} ( x , y ) = \frac{{Q}_{i n}}{2 π G} [ \begin{matrix}( \frac{( x - \frac{d}{2} )}{{( x - \frac{d}{2} )}^{2} + {y}^{2}} - \frac{α ( x - \frac{d}{2} )}{{( x + \frac{d}{2} )}^{2} + {y}^{2}} ) \hat{x} + \\ ( \frac{1}{{( x - \frac{d}{2} )}^{2} + {y}^{2}} - \frac{α}{{( x + \frac{d}{2} )}^{2} + {y}^{2}} ) \hat{y}\end{matrix} ] \qquad ( 2 )$$

The characteristic length of the dipole footprint (L_{D}) is given by Equation 3. (Safavieh et al., Sci Rep 2015; 5: 11943) (Qasaimeh et al., Nat Commun 2011; 2: 464)

$${L}_{D} = d ( \frac{α + 1}{α - 1} ) \qquad ( 3 )$$

The velocity profile and characteristic length equations for other microfluidic multipole configurations have similar forms. (Goyette et al., Nat Commun 2019; 10: 1781) (Qasaimeh et al., Nat Commun 2011; 2: 464) An important consideration in microfluidic-based manipulation and analyses of cells is to ensure that imposed shear stress does not exceed physiological limits. Hence, calibration of the probe 400 parameters for low shear stress is important. According to Stokes law, the shear stress imposed by the HFC is proportional to the tangential flow velocity (v ^{→}), see Equation 4, where η is the fluid's viscosity.

$$τ ( x , y ) = \frac{6 η}{G} \vec{v} ( x , y ) \qquad ( 4 )$$

Based on the selected aperture sizes and an electrode-substrate gap of 20 μm, the flow rates were numerically calculated with imposed shear stresses well below the “critical shear stress levels” of mammalian cells of about 1 Pa, see FIG. 12E. (Ludwig et al., Enzyme Microb Technol 1992; 14: 209-213) According to Equation 4, shear stress values can be further reduced either by increasing the tip-substrate gap or reducing the flow rate.

From Equations 1, 2 and 4, it is evident that the tip-substrate gap is an important parameter that controls the probe 400 electrical and fluidic manipulation parameters. This parameter has historically been controlled using visual techniques that are prone to human error. In this work, automated control of G was achieved using an image recognition technique based on Kernalized Correlation Filters (KCF). (Henriques et al., IEEE Trans Pattern Anal Mach Intell 2015; 37: 583-596) The procedure depends on tracking and measuring the horizontal displacement (HD) resulting from a downward vertical displacement (VD) when the tip of the electrode is in contact with the bottom substrate. Since the onset of HD coincides with the zero VD, VD was correlated with HD for a given probe holder stiffness and electrode/glass slide coefficient of friction. The experimental correlation between VD and HD were also numerically validated with a friction coefficient of 0.1841. Subsequently, zeroing of VD was achieved by tracking and measuring HD using the image recognition module, and using the deduced correlation to calculate VD. Upon zeroing VD, the Z axis of the XYZ micro-positioner 105 controller was used to position the electrode's tip at the desired tip-substrate gap. In order to facilitate this procedure, “zeroing areas” void of cells were reserved on the substrate and gentle lowering of the probe 400 was ensured to avoid abrasion of the electrode's tip.

In FIGS. 12A-12F an example experimental characterization of system 100 including probe 400 is shown. FIGS. 12A and 12B are a side sectional view and a bottom view, respectively, of the tip of the probe 400 showing the simultaneous confinement of an electric field and reagents to a single-cell target for nucleic acid transfer through the cytoplasm membrane.

FIG. 12C shows finite element modeling of the electric field contour (top) and electric field magnitudes across the substrate 110 (bottom). The plots show a high electric field confinement to areas directly beneath the electrode 445. For the plot shown, a 2 V potential was applied to an electrode-substrate gap of 20 μm.

FIG. 12D shows the velocity profile (left) and streamline (right) of the two apertures probe (top row) and four apertures probe (bottom row) fluid flow confinement with an inlet aperture flow rate (Qᵢₙ) of 0.25 μL/min and a flow ratio (Qᵢₙ/Qₒᵤₜ) of 2 obtained from finite element modelling.

FIG. 12E is a plot depicting shear stress experienced by target cells beneath the electrodes as a function of flow ratio with a constant outlet aperture flow rate (Qₒᵤₜ) of 0.5 μL/min.

FIG. 12F is a plot showing experimental correlations between the vertical displacement (VD) and horizontal displacement (HD) of the probe handle for the image recognition enabled tip-substrate gap manipulation. Error bars are standard deviation from 4 repeats. A finite element based model, with a glass-probe coefficient of friction of 0.18 was used as validation.

FIG. 13 depicts an electrical lysis experimental application utilizing the system 100 with probe 400. As shown in the figure, the probe 400 approaches the cell to be manipulated from above. In one embodiment the cell is still in its tissue organization. When the tip of the probe 400 is at the proper distance above the cell to be manipulated, the electrode 445 is activated to perform the electrical lysis application. The electric field produced by the electrode 445 creates pores on the cell membrane. The cytoplasm released from the sampled cell is then collected via the outlet aperture 455 via a negative pressure differential.

FIG. 14 depicts an oil droplet compartmentalized sequential collection experimental application utilizing the system 100 with probe 400. In the example shown, the inlet aperture 450 is connected inside the probe 400 directly an outlet flow conduit above the outlet aperture 455. As shown in the figure, the probe 400 approaches the cell to be manipulated from above. In one embodiment the cell is still in its tissue organization. When the tip of the probe 400 is at the proper distance above the cell to be manipulated, an electric field can be applied by the electrode 445. Additionally, a first oil droplet is provided by the inlet aperture 450 and a negative pressure differential can be established via the outlet aperture 455. Once the cell has been sampled and the cytoplasm has been collected, a second oil droplet can be provided by the inlet aperture to compartmentalize the sampled cell cytoplasm between the first and second oil droplets. The probe 400 can then be moved to a next cell to be sampled, and the process repeats itself to provide a second compartmentalized cell cytoplasm sample.

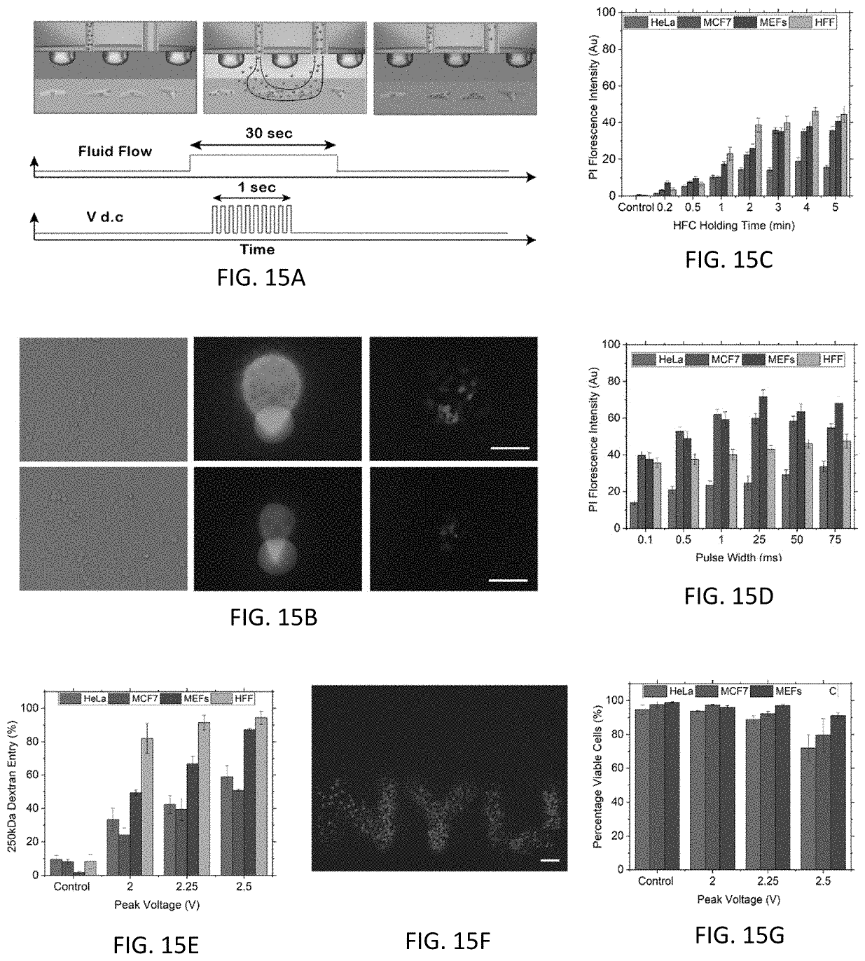

FIGS. 15A-15G depict an experimental application for the system 100 and prob 400 performing EP characterization of adherent cells using the system 100 with probe 400. The suitability of the probe 400 as a characterization tool for the transfer of extracellular molecules into adherent cells via EP was validated by delivering fluorescently labelled impermeable molecules of different sizes (propidium Iodide (0.68 kDa) and Fluoresceinisothiocyanate-dextran(250 kDa)) into mammalian cells. This demonstration relies on intracellular excitation of propidium iodide's (PI) and isothiocyanate-dextran (FITC-Dextran) fluorescence as an indicator of compromised cell membrane. (Weaver et al., FEBS Left 1988; 229: 30-34.) In order to expand the target area from single cells to a cluster of cells, the probe 400 configuration used for this characterization distribute multiple humps across the whole tip, see FIG. 15A. The electrodes 445 were positioned 20 μm above the target cells cultured on the counter electrode substrate and simultaneous activation of the HFC (PI dissolved in the EP buffer, Q_out=0.5 μl/min, 30 sec duration) and D.C. pulse parameters (2V, 10 Hz, rectangular pulses, 100 μs pulse width, 1 sec duration) resulted in transfer of PI into the target MCF-7 cells, as indicative of the cells labelled with red stains in FIGS. 15B and 15C. The ability to reduce the target footprint by simply adjusting flow ratios (Qₒᵤₜ/Qᵢₙ) from 4 (FIG. 15B) to 10 (FIG. 15C) was also demonstrated. Restriction of the resulting PI stained cells to the HFC footprint confirms confinement of the PI solution from the surrounding fluid.

Since EP pore sizes are dependent on pulse width, and diffusion of molecules through the cell membrane is time dependent, the pulse width and the HFC exposure time were selected as characterization parameters for the dosing rate. (Tsong, Electroporation and electrofusion in cell biology. Springer, 1989, pp 149-163) Due to the complexity of biological membranes, which include uneven surface charge distribution, non-uniform membrane thickness, and interference from intracellular cytoskeleton, accurate theoretical relationship between pore size and pulse-width are not feasible. Hence, experimental characterizations were relied on to obtain such relationship. As expected, these parameters regulated the created pore size on four different adherent cells as indicative of the increase in PI intensity with HFC exposure time and pulse width, see FIGS. 15C and 15D. The percentage entry (number of fluorescence cells/number of target cells) of macromolecules is also expected to be dependent on the applied field strength and this relation was characterized with 250 kDa FITC dextran molecules, see FIG. 15E. As a control, in the absence of electric field, minimal change in fluorescence intensity was observed which confirms the dependence of molecule intake on EP, see FIGS. 15C and 15E. An interesting finding was that the time provided for cell attachment to the ITO coated glass slides significantly affected delivery of PI. 24-hour cell attachment resulted in significantly increased dye intake compared to 3-hour cell attachment. This clearly highlights an increase in EP potential with increased cell stretching in adherence culture. While the exact dynamics are not fully understood, it is attributed to the reduction of folds and microvilli upon full adhesion/stretching which is expected to lead to an increase in permeability. Furthermore, to demonstrate spatiotemporal control, PI molecules were transferred into MCF-7 cells in a pattern that spelt “NYU”, see FIG. 15F. This highlights the advantage of the probe 400 as a dynamic tool with unprecedented spatiotemporal control for EP-based cell manipulation applications.

Application of high electric fields could lead to irreversible EP and subsequently cell death. If the threshold electric field strength is exceeded, the number and size of generated pores surpasses the acceptable limit and hence results in membrane rupture. (Weaver et al., Plant cell electroporation and electrofusion protocols. Springer, 1995, pp 3-28) (Weaver et al., Bioelectrochemistry Bioenerg 1996; 41: 135-160) As such, cell viability is an important indicator used to measure the success of reversible EP and the viability of the electrical parameters was validated by performing a live/dead assay on the target areas 24 hours after a 3-minute fluid exposure of plain EP buffer with no fluorescent dye, and 2V DC rectangular pulses of 50 ms width, see FIG. 15G. (Canatella et al., Biophys J 2001; 80: 755-764.) These HFC and electric pulse parameters were selected based on observed saturation of PI intake at these values, see FIGS. 15C and 15D.

Further description of FIGS. 15A-15G follows. FIG. 15A depicts the step-by-step schematics (top) and time trace (bottom) of EP based dye transfer into cells. With a distribution of the hump electrode across the probe 400 tip, all cells beneath the probe experience the electric field but only the cells confined by the HFC received the molecules.

FIG. 15B depicts the Bright field image (left), FITC fluorescence image during HFC activation (middle), and red fluorescence image after EP (right) of the target area within an MCF-7 culture for Qₒᵤₜ/Qᵢₙ=4 (top row) and Qₒᵤₜ/Qᵢₙ=10 (bottom row). Pulse width was 100 μs and HFC exposure was 30 secs. Scale bars are 50 μm.

FIG. 15C is a plot of the PI fluorescence intensity of target cells after simultaneous electroporation (100 μs) and exposure to PI reagent HFC with varying duration.

FIG. 15D is a plot of the PI fluorescence intensity of target cells after 3 minutes' exposure to the PI reagent HFC and 1 sec exposure to electric pulses of varying pulse width.

FIG. 15E is a plot showing the percentage entry of 250 kDa FITC dextran after 3 minutes' exposure to the FITC dextran HFC and 1 sec exposure to 10 Hz, 50 ms electric pulses.

FIG. 15F depicts a red fluorescence image of the scanned area after simultaneous exposure to the HFC (30 sec resident time) and 50 ms electric pulses while operating as a scanning probe. Scale bar is 50 μm.

FIG. 15G is a plot showing cell viability 24 hours after exposure to HFC of EB (3 minutes' exposure time) as a function of peak voltage (50 ms pulse width). Percentage viability drops below 90% at 2.5 V indicating the onset of the threshold value for the probe 400 while operating with a DC pulse. Error bars represent standard error from at least 40 cells and 4 repeats.

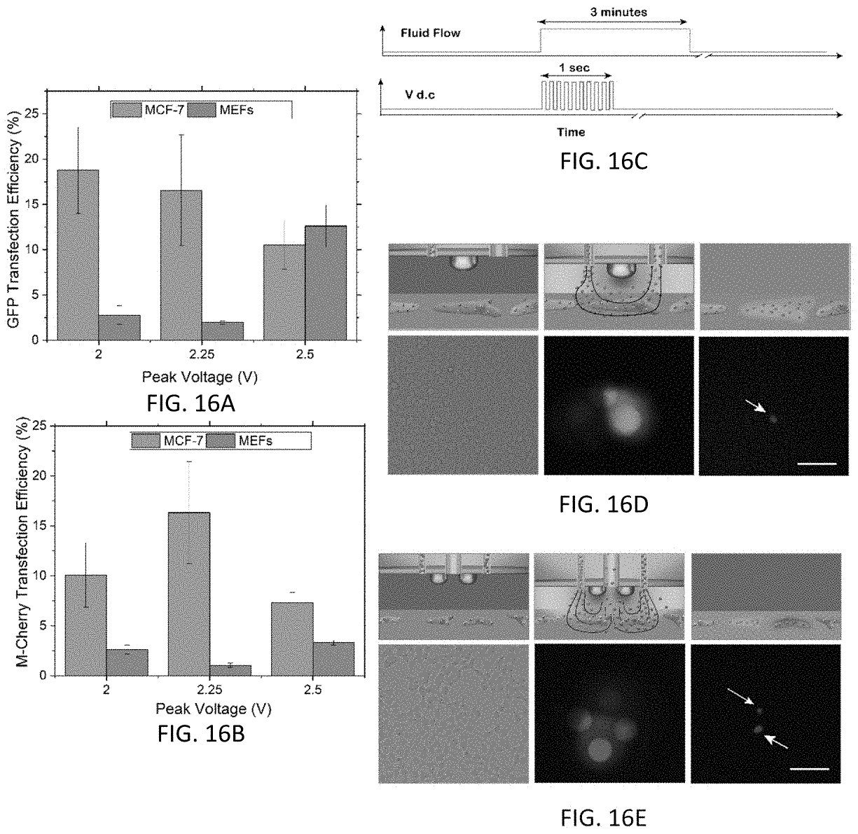

FIGS. 16A-16E depict an experimental application for the system 100 and the probe 400 performing multiplexed single-cell transfection. The approach of delivering genes as plasmid DNA vectors via EP is a potent alternative to the use of recombinant viruses. (Douglas et al., Biotechnol Prog 2008; 24: 871-883) Having established the capability to deliver molecules of different sizes into the cytoplasm, the probe 400 was used to deliver mammalian expression vector for green fluorescent protein (pCMV-GFP) to target cells within a culture of an adherent human cancer cell line (MCF-7). (Matsida et al., Proc Natl Acad Sci 2004; 101: 16-22) As expected, the target cell footprint bounded by the HFC precisely matched the transfected cell region, as indicated by the presence of green fluorescent MCF-7 cells, 24 hours after EP. The transfection efficiency was also characterized as the ratio of number of fluorescence cells to the number of target cells for MCF-7 and primary mouse cell (Mouse Embryonic Fibroblast (MEFs)). Transfection efficiencies between 2-20% were obtained for pCMV-GFP and monomeric red fluorescent proteins (mCherry2-C1) upon varying the pulse amplitude up to 2.5V, see FIGS. 16A and 16B, after which there is a decrease in viability as previously shown in FIG. 15G. These efficiencies are in-line with those obtained in previous similar studies. (Hass et al. Neuron 2001; 29: 583-591)

To validate the use of the probe 400 for single-cell targets, a monoplex electrode was used to concentrate the electric field to a smaller footprint. Successful transfer of PI into single-cell targets demonstrates the ability to perform single-cell transfection by simultaneous confinement of the pCMV-GFP reagent, with a 3-minute exposure time, and with the D.C. rectangular electric pulse to a single-cell, see FIG. 16C and top row FIG. 16D). An example representation in FIG. 16D shows a green fluorescence cell at the target area obtained 24 hours after processing with the probe 400, which confirmed successful single-cell transfection with pCMV-GFP plasmid. Such spatial resolutions to achieve single-cell transfection have also been previously achieved with microelectrodes and micro-pipettes. (Hass et al. Neuron 2001; 29: 583-591) (Judkewitz et al., Nat Protoc 2009; 4: 862) (Olofsson et al., Curr Opin Biotechnol 2003; 14: 29-34) However, since no fluid confinement is achieved by these devices, it is challenging to scale up for simultaneous delivery of different genes to neighboring multiple single-cells. To resolve this, the adapted 3D printing fabrication technique makes the probe 400 also easily adaptable for simultaneous multiplexed single-cell gene delivery. This was achieved by implementing the duplex electrode and microfluidic quadrupole configuration, see FIG. 7B, to simultaneously deliver two different plasmids to adjacent cells in the same vicinity, see top row of FIG. 16E. Visible green and red fluorescent single-cells confirmed isolated transfection of adjacent single-cells with the pCMV-GFP and mCherry2-C1 plasm ids respectively, see bottom row of FIG. 16E. The probe multiplexed transfection feature can be extended for an increased number of single cells by inserting additional electrodes and microfluidic multipole configurations. (Brimmo et al., Lab Chip 2019; 19: 4052-4063) (Juncker et al., Nat Mater 2005; 4: 622-627) (Qasaimeh et al., Nat Commun 2011; 2: 464)

Further description of FIGS. 16A-16E follows. FIG. 16A is a plot showing the pCMV-GFP transfection efficiency in MCF-7 and MEFs cells as a function of D.C. pulse amplitude. The electric pulse properties were 10 Hz frequency, 50 ms pulse width and 1 sec duration. The HFC properties were Qₒᵤₜ/Qᵢₙ=2 and exposure time of 3 minutes. Error bars represent standard deviation from minimum of 4 repeats.

FIG. 16B is a plot showing the mCherry2-C1 transfection efficiency in MCF-7 and MEFs cells as a function of D.C. pulse amplitude. The electric pulse properties were 10 Hz frequency, 50 ms pulse width and 1 sec duration. The HFC properties were Qₒᵤₜ/Qᵢₙ=2 and exposure time of 3 minutes. Error bars represent standard deviation from minimum of 4 repeats.

FIG. 16C depicts the time trace of the HFC and D.C. electric pulses used for validation single-cell transfections.

FIG. 16D depicts the step-by-step schematics of the single-cell transfection process (top row) and corresponding bright field (left bottom), FITC fluorescence image during the electroporation (middle bottom), and green fluorescence image 24 hours after transfection with PCMV-GFP plasmid (right bottom). This process was performed using the probe 400 configuration of FIG. 7A. The HFC in the middle bottom panel is labeled with green fluorescein sodium salt to aid visibility. The D.C. electric pulse properties used were 10 Hz frequency, 50 ms pulse width and 1 sec duration. The HFC properties were Qₒᵤₜ/Q=4 and exposure time of 3 minutes.

FIG. 16E depicts the step-by-step schematics of the multiplexed single-cell transfection process (top row) and corresponding bright field (left bottom), FITC fluorescence image during electroporation (middle bottom), and merged green and red fluorescence image 24 hours after transfection with pCMV-GFP and mCherry2-C1. (right bottom). This process was performed using the probe 400 configuration of FIG. 7B. The HFCs in the middle bottom panel are labelled with green fluorescein sodium salt for visibility and the top half is modified with a red filter on image. The D.C. electric pulse properties used were 10 Hz frequency, 50 ms pulse width and 1 sec duration. The HFC properties were Qₒᵤₜ/Q=4 and exposure time of 3 minutes.

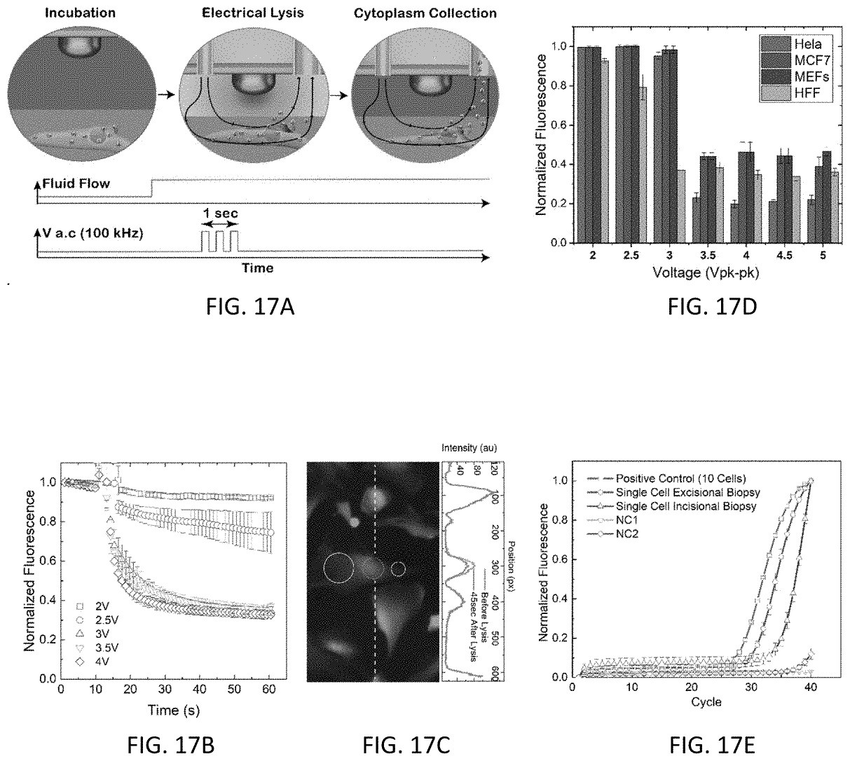

FIGS. 17A-17E depict an experimental application for the system 100 and probe 400 performing controllable single-cell biopsies. The ability to extract cytoplasmic contents from single-cells without dissociating them from their physiological environment provide fundamental omic insights while maintaining cellular and tissue integrity. The probe 400 enables non-contact cytoplasmic extraction (biopsy) from single cells by using a confinement of hypoosmotic EP buffer (Qₒᵤₜ/Q=4) to extract the cytoplasmic content of cells released via EP, see FIG. 17A. While EP is commonly performed with D.C. pulses, A.C. electric signals provided better confinement of the generated electric field and hence more suitable for releasing cytoplasmic contents. This is attributed to favorable membrane dynamics in the presence of a frequency of a 100 kHz AC field.

Target cells were first pre-labelled with calcien-AM, and cytoplasm extraction was monitored by reduction in fluorescence intensity. Demonstrations on the probe's fine spatial precision for targeted single-cell biopsies was then performed on primary Human Foreskin Fibroblast (HFF) cells. During characterizations, it was observed that the cytoplasm extraction rate, indicated by the rate of reduction of calcein intensity, is dependent on the applied peak-to-peak voltage, see FIG. 17B. This can be attributed to the expected increase in pore size with voltage. Such characteristics translate to a fine control on the pore sizes in order to either perform reversible EP to extract only a portion of the cell's cytoplasmic content at ≤2.5 V peak-to-peak (incisional cell biopsy) or completely lyse the cell to collect all of its cytoplasmic content ≥3 V peak-to-peak (excisional cell biopsy). For incisional biopsies of HFF cells, the calcein intensity after 5 minutes of EP shows an average of 25%±10.5% drop, see FIG. 17B, with a representative image shown in FIG. 17C. Visual inspection suggested that the integrity of cells that underwent incisional biopsies was maintained so the possibility of performing multiple biopsies at different time points was further assessed. In this case, two biopsies were carried out 2 h apart from each other and cell viability, which was monitored for up to 24 h, was not significantly affected. This enforces the suitability of the probe 400 for performing spatiotemporal resolved single-cell transcriptomic and proteomic analysis, where multiple manipulations can be performed at different time points during the cell developmental process.

In addition, the applicability of the controllable biopsy technique for a range of cell types which include human cancer cell lines and primary mouse embryonic cells, and observed similar trends as with HFF cells was demonstrated, see FIG. 17D. Excisional biopsy of HFF cells were also validated visually. Upon extraction of all of the cell's cytoplasm, it was observed that the cell nucleus remains attached to the substrate. This indicates the feasibility of using the probe 400 to separate cells' cytoplasmic and nuclear contents as a basis for applications on probing physiological triggers for varying gene expressions in the sub cellular compartments. (Abdelmoez et al., Genome Biol 2018; 19: 1-11)

The low volume of extracted single-cell cytoplasm (approx. 0.1 pL) necessitated significant dilution for subsequent retrieval with conventional serological pipettes. As such, in order to validate detectability via quantitative polymerase chain reaction (qPCR) amplification, the housekeeping RNase-P RNAs with TaqMan probes and primers were targeted. Reverse transcription into complementary DNA (cDNA) was done using a thermal cycler and this cDNA was amplified using qPCR. Samples from the HFF excisional biopsies reached the cycle threshold (Ct) value after an average of 30.8±0.3 cycles while incisional biopsies samples had an average Ct value of 35.4±0.2, see FIG. 17E. These suggest that the integrity of sampled RNAs are retained and detectable by qPCR amplification, hence validating the suitability of the probe 400 for single-cell transcriptomic and proteomic sampling. As positive control, the RNase-P RNAs contents from 10 HFF cells preprocessed by chemical lysing and obtained an average Ct value of 28.4±0.1 was quantified. ΔCt between excisional biopsies (1 cell) and the positive control (10 cells) is 2.4, which translates to a measured dilution of 5.27—assuming 100% doubling of DNAs per cycle. This is less than expected but represents a reasonable error range considering difference in protocols and heterogeneity of single cells. EB sampling using the probe's HFC before the application of the electric field was used as the first negative control (NC1) and no traces of the targeted gene was found after 50 cycles (undetermined Ct). A second negative control (NC2) was collected 5 minutes after cell biopsies to confirm complete extraction at a later time point, and only traces of the RNase-P RNAs was detected with an average Ct of 40.2±0.4.

Further description of FIGS. 17A-17E follows. FIG. 17A depicts a step-by-step schematic (top) and time trace (bottom) for the single-cell biopsy operation of the probe 400. The operation begins with an incubation, followed by electrical lysis where the HFC and the electric fields are applied by the probe 400. The operations ends with continued HFC to perform cytoplasm collection.

FIG. 17B is a plot showing normalized FITC fluorescence intensity of HFF before (0-15 secs) and after (15-60 secs) cell biopsies, as a function of applied peak-to peak voltage (100 kHz fq, 1 sec duration). Error bars indicate a standard deviation from 4 individual measurements.

FIG. 17C depicts a green fluorescence image of calcein-AM labelled HFF cells after incisional cell biopsies. The plot at the right side represents the spatial fluorescence intensity across the dotted line sections to quantify the green intensity drop in the target cell and show that intensities of neighboring single-cells do not drop after targeted incisional biopsies. White dotted circles represent the positions of the inlet aperture 450 and outlet aperture 450 while the red dotted circle represent the position of the hump electrode 445. The diameter of the outlet aperture is 60 μm.

FIG. 17D is a plot showing normalized FITC intensity of target HeLa, MEFs, MCF and HFF cells 1 minute after single-cell biopsies as a function of peak-to-peak voltage. Error bars indicate standard deviations from 4 individual measurements.

FIG. 17E is a plot showing the mean qPCR amplification curve for RNase P RNAs for incisional and excisional HFF biopsies along with positive controls (10 cells preprocessed with chemical lysis), negative controls (NC 1-probe sampling before biopsy and NC 2—probe sampling 5 minutes after biopsy). Error bars indicate standard deviation of 4 individual measurements.

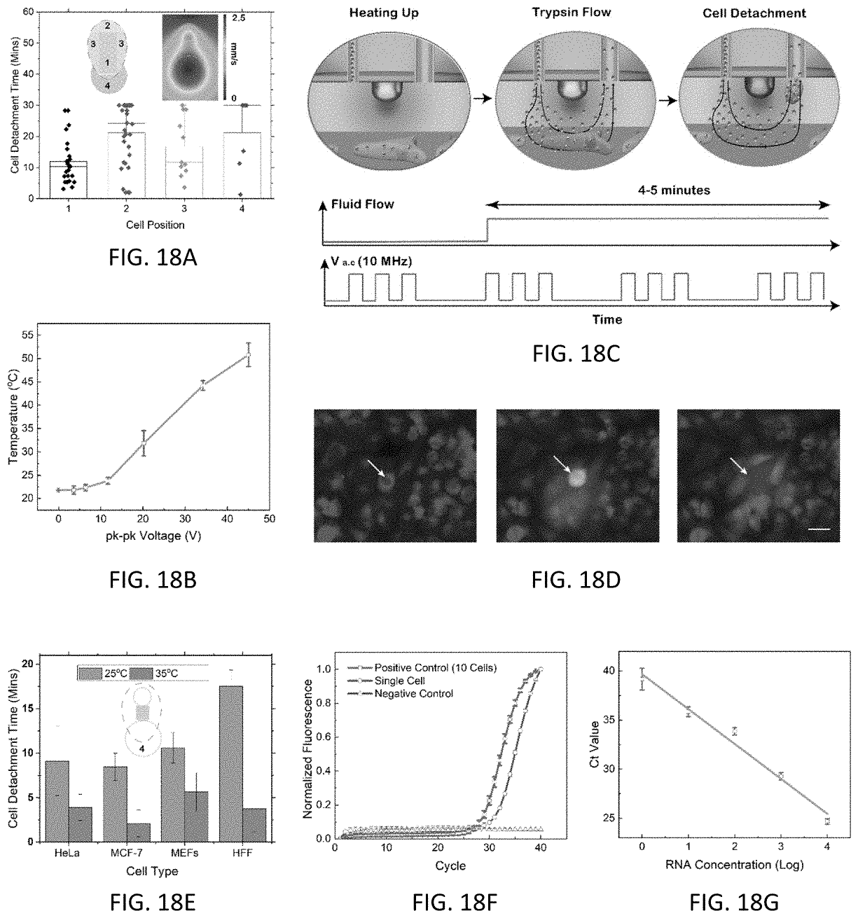

FIGS. 18A-18G depict an experimental application for the system 100 and probe 400 performing heat assisted single whole cell dissociation, also known as a heat assisted single-cell tweezer. The full scope of single-cell omics involves the analyses of the genomes, transcriptomes and proteomes. While transcriptomes and proteomes information can be retrieved from the cell's cytoplasm, genomes are predominantly concentrated within the nucleus. The experimental demonstrations of the probe 400 based single-cell biopsies show that the nucleus is retained on the cell substrate after cytoplasm extraction. In order to subsequently retrieve contents of the nucleus along with the cytoplasm, whole single-cell enzymatic dissociation was utilized to retrieve an intact whole cell, and hence the nucleus containing the genomes information. Selective detachment and collection of a single living cell from a surface, using enzymatic dissociation, has been previously demonstrated by the microfluidic dipole. (Juncker et al., Nat Matter 2005; 4:622-627) In this approach, an MFP was used to flush a solution of trypsin over an adherent fibroblast cell to collect them in about 8 minutes. However, since at room temperature, trypsin activity is 1.78 times lower than at 35.5° C., there's room for optimizing this process by simultaneous localized heating of the trypsin reagent. (Trypsin, In: Methods of enzymatic analysis. Elsevier, 1974, pp 1013-1024)

In order to fully characterize the enzymatic dissociation process of the microfluidic dipole, the positional bias of the trypsin-HFC in retrieving single-cells at room temperature was first assessed. With this characterization, the trypsin HFC selectively dissociates HeLa cells with preference given to cells located between the inlet and outlet apertures (Area 1)—coinciding with the maximum flow velocity region, see FIG. 18A. The average cell dissociation time in Area 1 was about 10 minutes, which closely correlates with previously reported values. (Juncker et al., Nat Matter 2005; 4:622-627) In order to optimize this process, and hence reduce the dissociation time, a subtle change in applied voltage was implemented to impose Joule heating, see Equation 5, where ΔT is the temperature rise and a is the electrical conductivity of the solution.

ΔT∝σE² (5)

For this application, the A.C. frequency was increased to 10 MHz to avoid EP when high voltages are applied. (Menachery et al., IEE Proc-Nanobiotechnology 2005; 152: 145-149) A validation of the probe 400 to controllably increase temperature of the trypsin-HFC, based on Joule heating, is presented in FIG. 18B. By simultaneously confining single-cells with an HFC of trypsin Qₒᵤₜ/Qᵢₙ=4 and an A.C. signal (10 MHz, 22 V pk-pk), a fluid temperature of 35° C. was achieved to optimize dissociation of single-cell targets, see FIGS. 18C and 18D. The localized heat generation reduced the average dissociation time of HeLa cells by 43.3% and demonstrated similar time improvements in MCF-7, MEFs and HFF cells, see FIG. 18E, with no significant impact on the viability of cells retained on the culture substrate.

Upon retrieval, it is important that the cell remains intact to shield the cell contents from trypsin, in order to avoid possible trypsin digestion of internal proteins. The integrity of the cell membranes was assessed by performing reverse transcription and qPCR amplifications of target RNase-P RNAs in the collected single HeLa cell. These resulted in an average Ct value of 31.8±0.08 for RNase-P from retrieved single HeLa cells, see FIG. 18F. As positive control, the same procedure for 10 HeLa cells isolated via serial dilution was performed and the average Ct obtained was 29.2±0.4. Growth media within the culture dish of the dissociated cell was used as negative control and no target RNA was detected after 50 cycles (undetermined Ct). The validity of these qPCR quantifications was confirmed by obtaining a linear range curve from serial dilution of cDNAs and obtaining a PCR efficiency of 91% and R² value of 0.97961, see FIG. 18G. This enforces the ability of the probe 400 to maintain the membrane integrity of retrieved cells, and hence validates the tools suitability for retrieving transcriptomic, proteomic and genomic materials from single adherent cells.

Further description of FIGS. 18A-18G follows. FIG. 18A is a plot showing spatiotemporal characterization of trypsin detachment of HeLa cells at room temperature with no heat assistance. The spatial segmentation (top left), and finite element modelling based flow velocity (top right) are also shown.

FIG. 18B is a plot showing validation of the probe's 400 applicability in adjusting cell media temperature. Based on Joule heating, the temperature of the surrounding fluid can be adjusted by varying peak-to-peak AC voltage at a frequency of 10 MHz. The error bars indicate a standard deviation of 4 different trials.

FIG. 18C depicts a step-by-step illustration (top) and time trace (bottom) of the probe 400 manipulation application as a heat assisted single-cell enzymatic dissociation tool. The application begins with the probe being positioned above the targeted cell followed by the electrode 445 heating the surrounding volume. The trypsin flow is then initiated flowing from the inlet aperture 450 to the outlet aperture 455 leading to cell detachment and sampling of the cell.

FIG. 18D shows corresponding fluorescence images of Hela cells during the heating up step (left), 2 minutes after introduction of HFC (middle) and after single-cell tweezing (right). The HeLa cells were stained with cell tracker red prior to the experiments to aid visibility. White arrow points to the target cell, and the scale bar is 50 μm.

FIG. 18E is a plot showing that the single-cell detachment time was significantly reduced at 37° C. based on the optimized trypsin conditions. The target location was also optimized by focusing on the region depicted by the green marking in the inset.

FIG. 18F is a plot showing the mean qPCR amplification curve for RNase P RNAs for a tweezed single HeLa cell along with positive control (10 cells preprocessed with chemical lysis), and Negative Control (1 probe sampling before tweezing). Error bars indicate standard deviation of four individual measurements.

FIG. 18G is a plot showing the linear range of the qPCR assay based on serial 10× dilution of HeLa cells. The slope is −3.56 and the R² value is 0.979. Error bars indicate standard deviation of four individual measurements.

Further description of FIGS. 19A-19B follows. In some applications, the use of an ITO coated substrate configured as a counter electrode is not available. A probe including 2 or more electrodes can be configured for these applications, where at least one electrode is configured as a counter-electrode. This allows cell biopsy operations to be performed on any substrate, such as well plates, microtiter plates, and petri dishes, for example. The electrical isolation of the electrode and counter-electrode can be implemented via 3D printing a mask that can then be clipped off after fold sputtering, for example. FIG. 19A shows schematics and a photograph of the device including 2 or more electrodes can be configured for these applications, where at least one electrode is configured as a counter-electrode functioning inside a microtiter plate. FIG. 19B shows micrographs of the device showing two electrodes located at the tip of the probe. The SEM image on the right, shows probes and microfluidic apertures for HFC. Having both working and counter electrodes integrated on the MFP's tip allow working with cells cultured any nonconductive substrate.

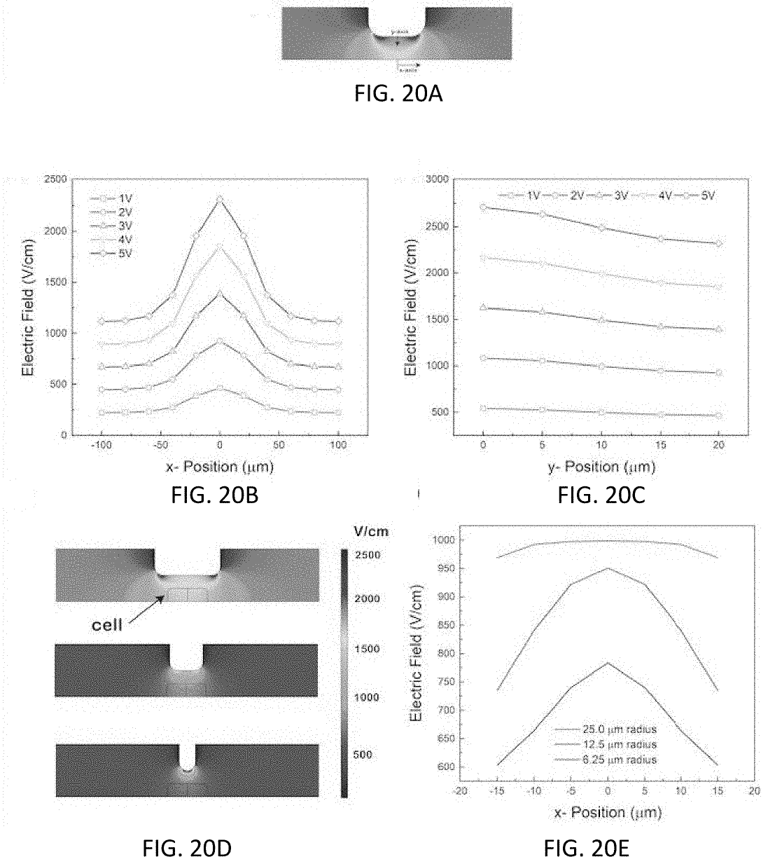

Further description of FIGS. 20A-20E follows. FIG. 20A shows the electrical field contour around the electrode calculated by finite element modeling. FIG. 20B shows the magnitude of the electric field as a function of applied voltage and the position on the bottom substrate. FIG. 20C shows the magnitude of the electrical field as a function of the applied voltage and the distance from the electrode at the zero x position. FIG. 20D shows the electric field contour as a function of electrode size. Electrodes with radius' of 25 μm (top), 12.5 μm (middle) and 6.25 μm (bottom) are positioned 20 μm away from the bottom substrate with a cell represented by a 15 μm radius and 10 μm thick cylinder positioned in between. FIG. 20E shows the magnitude of the electric field experienced at the top surface of the cell. The 25 μm radius electrode produces a uniform electric field across the cell's surface while those of 12.5 μm and 6. 25 μm produces the maximum electric field at the position corresponding to the center of the electrode.

Isolation of the electric field was achieved using an integrated pin-plate electrode configuration. In the experimental case, the pin electrode was a 25 μm base radius and 25 μm height cylindrical “hump” with 10 μm radius fillets on the tip, and the plate electrode was an ITO-coated glass slide. Shown using finite element modeling, the configuration can isolate high electric fields to the target area directly beneath the electrode (FIG. 20A). The magnitude of the field can be varied by adjusting the applied voltage but regardless of the applied voltage, the high electric field is concentrated within the 50 μm span of the electrode (FIG. 20B). The electric field magnitude also drops across the y-direction as the distance from the electrode increases. However, for a 20 μm electrode-substrate spacing, the maximum drop is approximately 15% at the zero x-position (FIG. 20C).

The electrode's diameter also affects the magnitude and distribution of the electric field on the substrate. As the electrode size reduces, concentration of the electric field at the electrode's tip increases and this reduces the magnitude of the electric field experienced by target cells (FIG. 20D). Reduced electrode size and hence increased electric field concentration also results in exposure of the cell's cross-sectional area to a non-uniform electric field magnitude (FIG. 20E). Hence, electrode tips less than 25 μm radius are expected to open pores in only the sections of the cells directly beneath the electrode and are hence more beneficial if the intention is to deliver molecules to specific target sections of the cell. In order to open pores across the whole surface of a single cell, and hence increase the chances of molecule delivery to the whole cell, electrode sizes that span across the whole diameter of an average single cell are recommended.

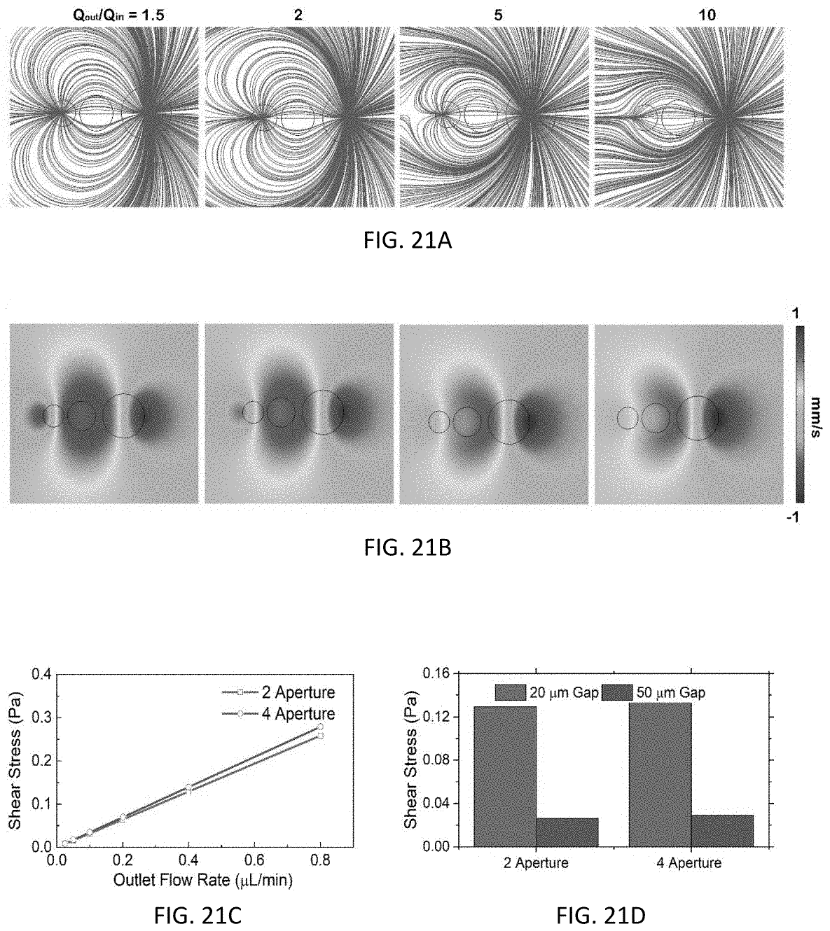

Further description of FIGS. 21A-21D follows. FIG. 21A shows the flow streamline as a function of Qout/Qin. FIG. 21B shows the flow velocity contour as a function of Qout/Qin. All figures are results of finite element modelling with a constant Qout of 0.5 μL/min. FIG. 21C shows the calculated relationship between shear stress and flow rate for Qout/Qin=4. FIG. 21D shows the calculated relationship between shear stress and electrode-substrate gap.

Hydrodynamic flow confinement (HFC) was achieved by simultaneous injection and aspiration through the individual apertures. For a given tip-substrate gap, the ratio of the outlet to inlet aperture flow rates (Qout/Qin) defines the confinement area (FIG. 21A). The fluid velocity at the target area is also affected by Qout/Qin as shown in the velocity contours of FIG. 21B. According to Stokes law, the flow velocity is proportional to the shear stress (τ) imposed on the cell which is given by Equation 4. From the equation, τ is directly proportional to the tangential velocity and inversely proportional to g. Hence for a given Qout/Qin, the shear stress experienced by the target cell is dependent on the flow rate magnitude (FIG. 21C) or the gap g (FIG. 21D).

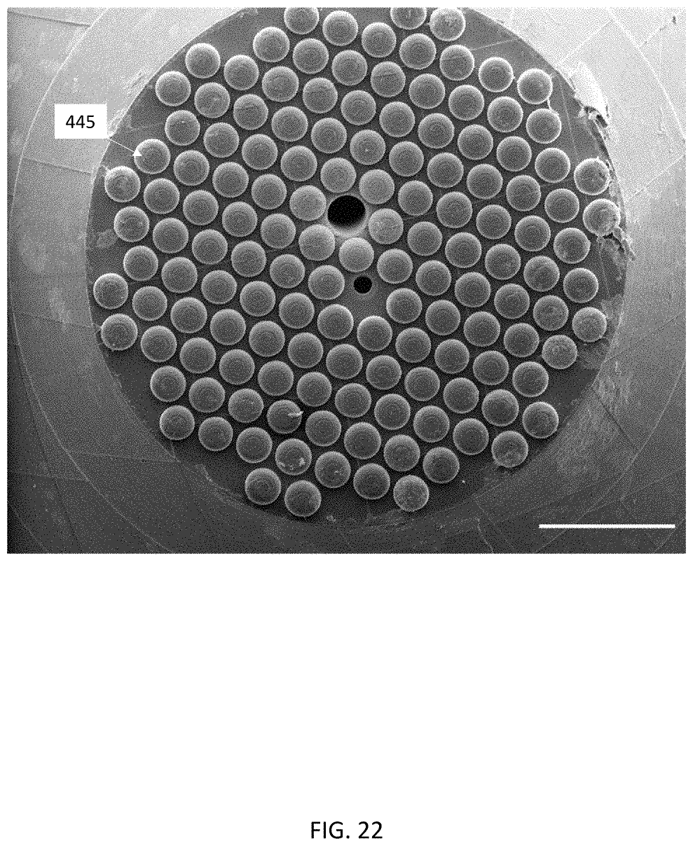

FIG. 22 shows an SEM image of an example experimental MMFP with a broad span electrode. The scale bar is 200 μm. In order to generalize the characterization process, a broad-span electrode version of the MMFP was developed by utilizing the entire surface of its tip as an electrode. This was achieved by distributing 138 hump shaped electrodes 145 across the whole tip.

Further description of FIGS. 23A-23D follows. FIGS. 23A-23D show the brightfield (top row), PI fluorescence (middle row) and FITC dextran fluorescence for HeLa (FIG. 23A), MEFs (FIG. 23B), MCF-7 (FIG. 23C), and HFF cells (FIG. 23D). A Qout/Qin=2, 3 min HFC holding time, and 50 ms pulse width is used for all experiments. Scale bar is 100 μm.

By dissolving PI and FITC Dextran into the EP buffer, simultaneous transfer of both molecules into target cell clusters was achieved. FIGS. 23A-23D show fluorescence mappings of the applied HFC as a function of the peak voltage. The first panel in the image clusters are control experiments where the HFC is applied for a duration of 3 min in the absence of the electric field. Minimal to no PI and FITC dextran fluorescence in these panels demonstrate dependence of the molecule intake on the electric field—with the observed minimal fluorescence attributed to staining of non-viable cells within the culture.

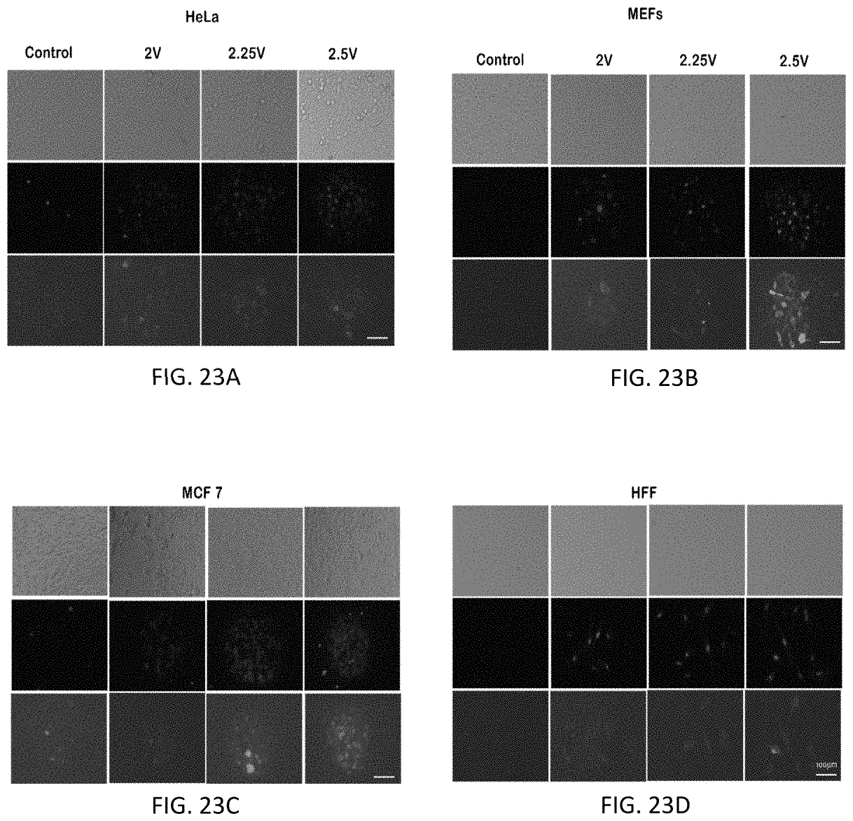

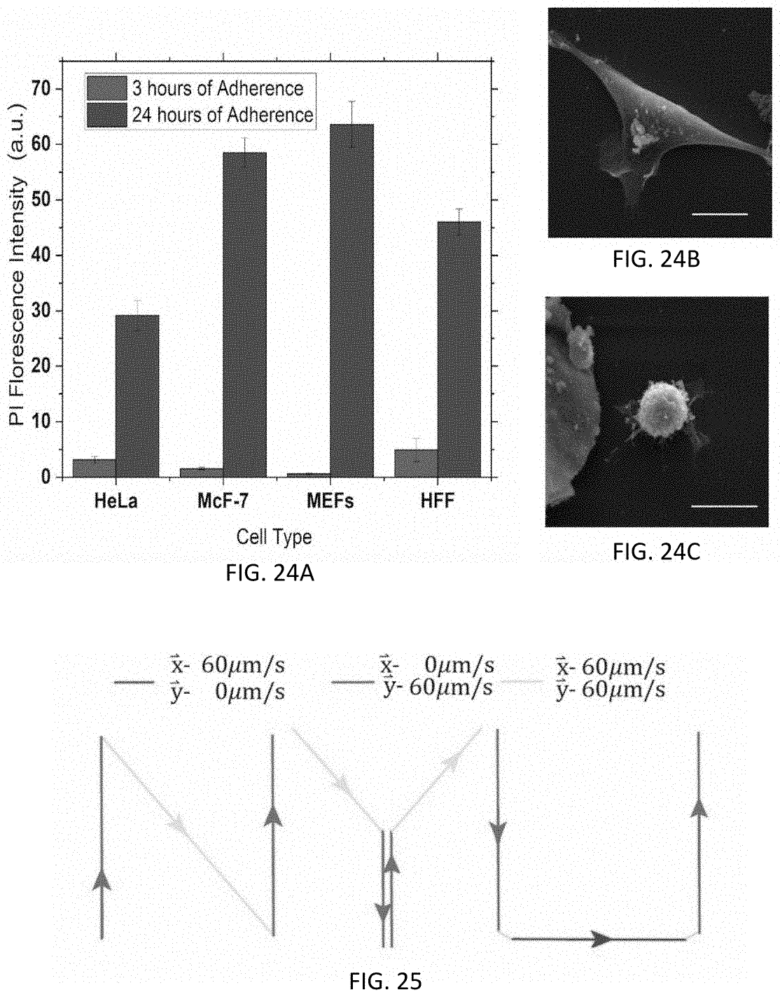

Further description of FIGS. 24A-24C follows. FIG. 24A shows the PI intake into cells as a function of time allowed for cell adherence as an indication of a comparison between the EP potential of cells in the adherent and suspended states. Error bars are standard deviation from individual measurements taken on 4 single cells. FIG. 24B shows the scanning electron microscope image of a representative MCF-7 cell after 24 h of adherence. FIG. 24C shows the scanning electron microscope image of a representative MCF-7 cell after 3 h of adherence. The visible presence of folds increases the effective thickness of the cell membrane to reduce increased displacement of electrical stresses. Scale bars are 10 μm.

The close proximity between the MMFP's electrodes, and the positioning of the target cells relative to the electrodes, permitted the use of low voltage magnitudes to achieve high electric fields. This is the basis behind achieving intake of macromolecules at applied signal amplitudes as low as 2 V. In addition, EP of cells while in adherent culture allowed for an easier membrane penetration in comparison to suspended cells. This was validated by comparing the PI intake at early and advanced stages of adherence. Using the same EP configuration for cells in both stages, increased PI intensity is observed after 24 h of adherence, in comparison to cells allowed to adhere for only 3 h (FIG. 24A). This can be attributed to the high-tension state and simplified morphology of adherent cells (FIG. 24B) as opposed to the abundant folds and microvilli presence in the suspended state (FIG. 24C). The reduction of excessive cell membrane area allows for less displacement of electrical stresses leading to higher membrane permeability of stretched lipid bilayers.

FIG. 25 shows an example experimental programmed scanning path and speed for the PI staining with “NYU” pattern. The scanning operation used to create PI intake patterns depends on the individual movement of the micro positioner's X and Y axis to create horizontal or vertical lines respectively, or simultaneous movement of the X and Y axis to create slanted lines. These line types were combined to form the desired “NYU” pattern based on the programmed axis movement scheme shown in FIG. 25.

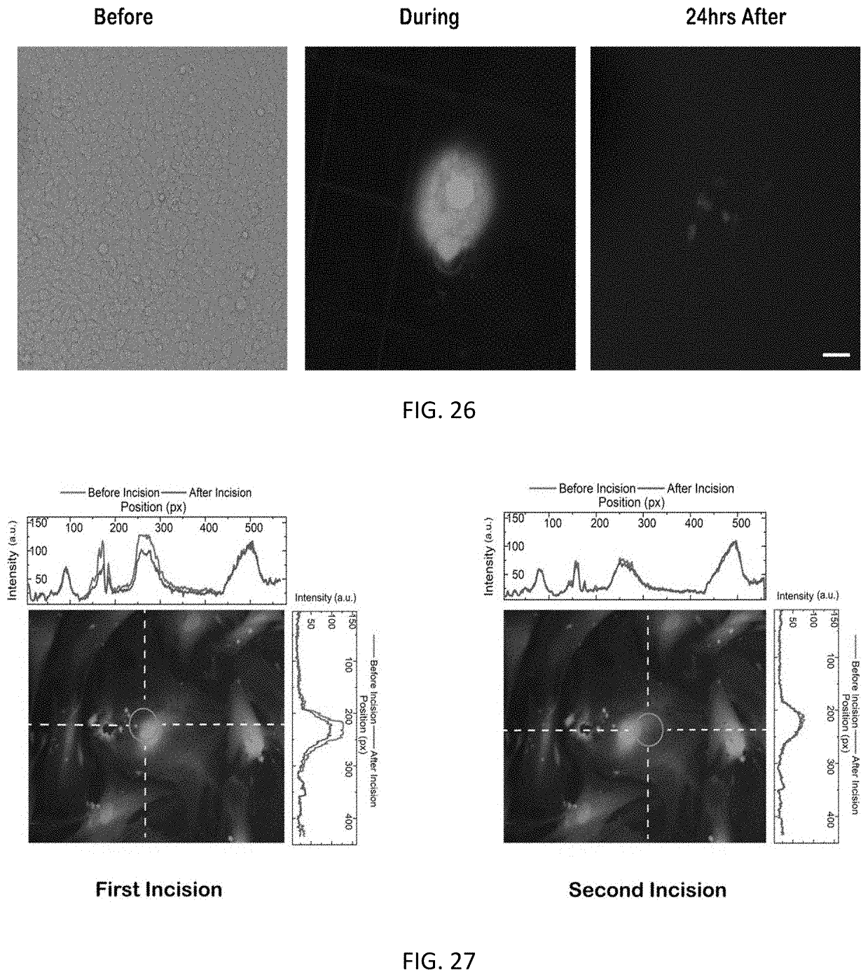

FIG. 26 shows the bright field (left), FITC fluorescence during transfection (middle) and FITC fluorescence 24 h after transfection (right) of a cluster of MCF-7 cells with the PCVM-GFP vector. Scale bar 50 μm.

To establish proper genetic functionality of the cells targeted with the broad-span electrode, transfection of MCF-7 cells with a plasmid vector was demonstrated with a green fluorescence protein (PCMVGFP). The plasmids are delivered by substituting the processing fluid with a mixture of the PCMV-GFP plasm ids and the EP buffer. After simultaneous exposure to the HFC (Qout/Qin=4) and the electric field (2 V DC, 10 Hz, 50 ms pulse width, 1 s duration), the cells were returned to the cell culture incubator. FITC fluorescence images after 24 h of incubation shows transfection of about 20% of the target cells. This low transfection efficiency can be attributed to the high degree of variability in the physiological conditions of the cells in adherent culture.

FIG. 27 shows green fluorescence images of HFF cells after multiple time point incisions. Incisional biopsies were made 2 h apart with 2.5 Vpk-pk, 100 kHz AC signals applied for a 1 s duration. The white dotted lines represent cross sections at which the inset intensity signals are collected and the orange circle represents the position of the electrode.

Incisional biopsies were used to carry out multiple sample extraction at different time points, and different locations, from the same cell. In this example, two biopsies were performed 2 h apart at different membrane locations. Prior to the experiment, cells were stained with calcein AM to monitor permeability and viability in real time. Following each incision, fluorescent signals across sections of the cells were used to estimate the quantity of extracted cytoplasm content and verify localization of the biopsy. After the second biopsy, the cell was found to retain calcein AM fluorescence and ruled out any significant cell damage. The fluorescence intensity drop after the first incision is significantly higher than that of the second incision and which is attributed to a higher concentration gradient of calcein AM in the first incision.

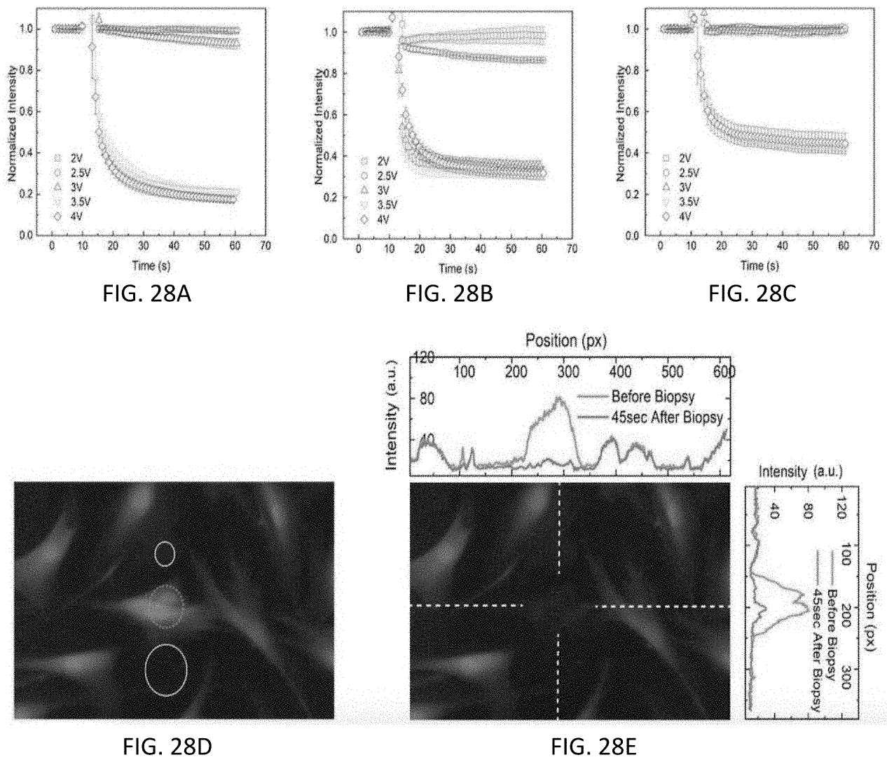

Further description of FIGS. 28A-28E follows. FIGS. 28A-28E show real-time monitoring of normalized fluorescence intensity during and after the MMFP based single cell biopsy of HeLa (FIG. 28A), MCF-7 (FIG. 28B), and MEFs cells (FIG. 28C) with a Qout/Qin=4, AC frequency of 100 kHz and duration of 1 s. Green fluorescence images of HFF cells before (FIG. 28D) and after (FIG. 28E) excisional biopsies using the MMFP are also shown. Plots on the top and left side of FIG. 28E represent quantitative fluorescence intensity across the vertical and horizontal sections highlighted with dotted white lines. The applied electric field in e is a 3.5 V AC wave with 100 kHz frequency and 1 s duration.

Based on real-time tracking of calcein AM fluorescence intensity, it was observed that the transition from incisional to excisional biopsy also falls between 2.5-3 Vpk-pk for HeLa, MCF-7 and MEFs cells (FIGS. 28A-28C). However, after incisional biopsies, the average normalized intensity, which is an indication of sampled cytoplasm content, varies with cell types. This can be attributed to the physiological differences between the membranes of different cell types.

Excisional biopsies were performed on HFF cells and the fluorescence intensity of calcein AM was used to monitor membrane permeability (FIGS. 28D-28E). After performing excisional biopsy, calcein AM intensities were observed to drop to levels matching the background intensity of the substrate (inset FIG. 28E). Based on this, it is inferred that all of the cytoplasmic content has been released and the cell is hence no longer viable.

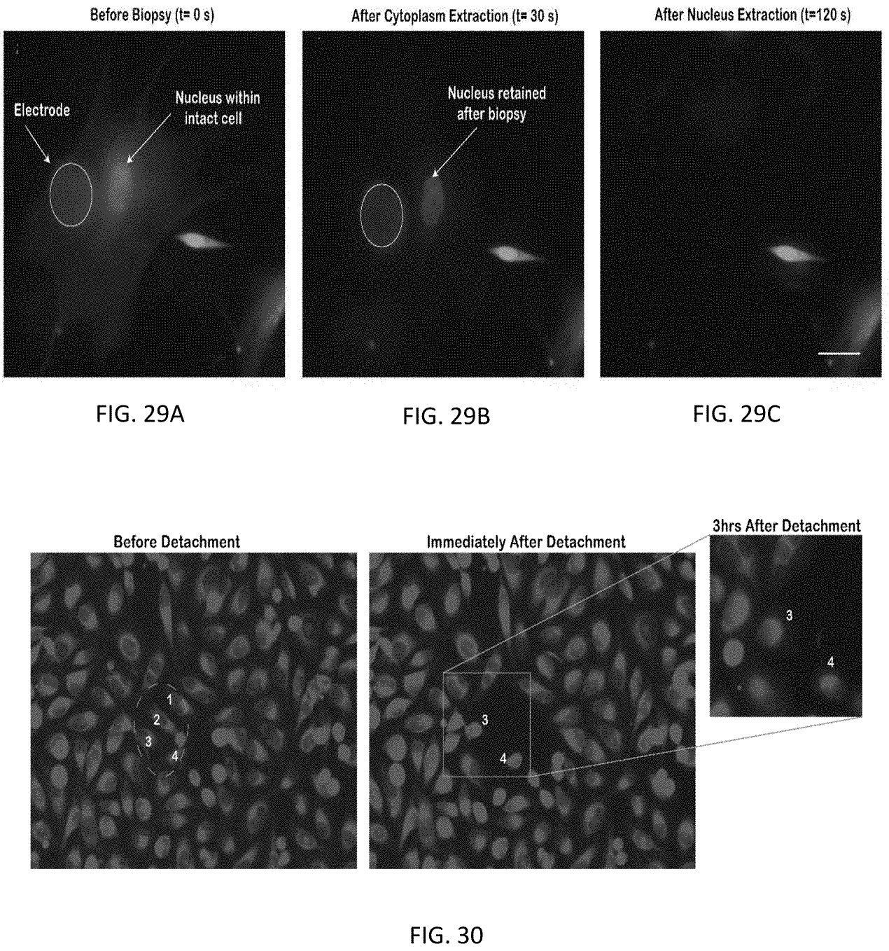

Further description of FIGS. 29A-29C follows. FIGS. 29A-29C show separate extraction of single-cell cytoplasm and nucleus. FIG. 29A shows the target cell before excisional biopsy. Green fluorescence is calcein-AM based representation of intact cells. FIG. 29B shows after excisional biopsy, the cytoplasmic content of the target cell is released and collected by a dedicated aspiration aperture. PI within the culture media stains the nucleus that remains attached to the substrate. FIG. 29C shows the lysed cell with retained nucleus retrieved by physical electrode detachment and hydrodynamic pull. Scale bar is 30 μm. The applied AC electric signal was with 3.5 V, 100 kHz, and 1 s duration.

After excisional biopsy, the cells cytoplasmic content is released to a dedicated aspiration aperture, but the nucleus remains attached to the substrate as indicative of the red PI stain. This suggests applicability of the MMFP for isolating subcellular compartments of single-cells. The electrode can then be used to physically detach the adherent membrane in order to ease nucleus retrieval from the substrate via hydrodynamic pull of an aspiration aperture. Alternatively, the retained nucleus can be subsequently characterized or sampled using other equipment such as the atomic force microscope for nanomechanical characterization.

FIG. 30 shows the recovery and viability of cells covered by the HFC of Trypsin. Cells 1-4 were confined by the HFC (left) and while the retained cells (3 and 4) show partial dissociation due to interaction with trypsin (middle), they begin to spread out again 3 h after incubation (right).

In a confluent culture, the HFC created from a 15 μm radius inlet aperture spanned across multiple cells. As such, other cells within the span of the HFC were partially affected by the dissociating effect of trypsin. However, these cells were observed to remain on the substrate and demonstrated recovery of their original adherent morphology after 3 h.

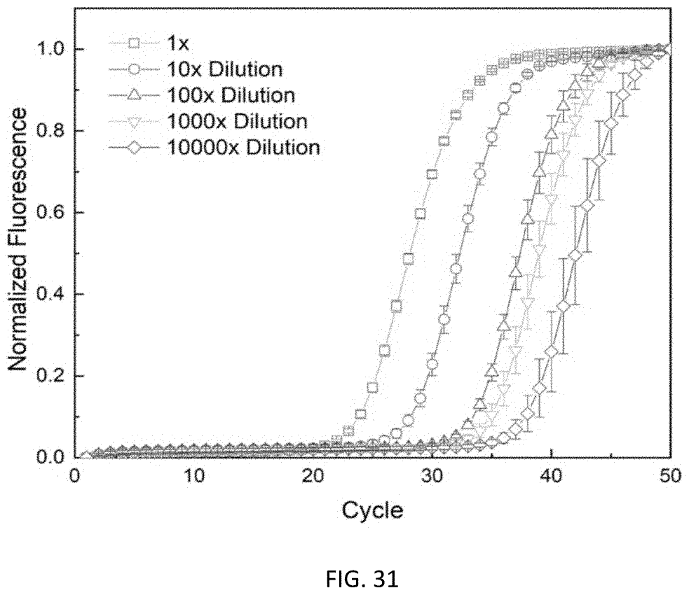

FIG. 31 shows the mean qPCR amplification curve for linear range plot. Error bars indicate standard deviation from 4 different measurements.

In summary, image recognition models and validated structural mechanics correlations were applied to enhance manipulation precision. The system's 100 utility was validated in performing electropermealization-based molecular transfer into cells in an adherent culture and performed comprehensive characterizations of the process. Accurate spatial control was demonstrated by precisely patterning a targeted cell cluster with an otherwise impermeable dye. Also demonstrated was multiplexed genetic manipulation in tissue-like cultures by transfecting adjacent single-cells with different DNA plasmid vectors. In addition, controllable cytoplasm extraction from living single cells without compromising the integrity of the extracted RNA or affecting cell viability was shown. Subsequent to the transfer of nucleic acids through the cell membrane, the probe's use was validated in facilitating temperature dependent enzymatic dissociation of single cells.

The multiphysics utility of the resulting probe 400 was demonstrated by transferring varying size exogenous molecules into cells in adherent tissue-like culture via EP. This enabled a comprehensive characterization of the EP-based molecular transfer response of cells in adherent culture. In addition to this, the spatial precision of the probe's electro-fluidic fields by precisely patterning an adherent cell cluster with impermeable dyes while operating in the scanning mode was validated. Furthermore, spatiotemporal genetic manipulation features by transfecting multiple single cells within the same vicinity, with different DNA plasmid vectors, was demonstrated. Also demonstrated was controllable extraction of the cytoplasmic content of single cells, without affecting the integrity of the extracted RNA or cell viability. Finally, with a subtle alteration of the electrical configuration, the probe 400 was used to facilitate heat dependent enzymatic dissociation of whole single cells for a complete omics analysis.

The concept behind the disclosed multiplexed single-cell genetic manipulation is well suited for simultaneous excitation of neurons in vivo. This facilitates an elaborate manipulation of gene expressions within complex neuronal circuits in order to mimic complex brain functions, and hence link knowledge of single neurons with theories of network functions. (Judkewitz et al., Nat Protoc 2009; 4: 862) Furthermore, the biopsy techniques disclosed present a unique approach for collecting multiple samples at different time points, while retaining the original spatial constraints. This could serve as an invaluable tool for increasing the general knowledge base on organ developmental processes like cardiac morphogenesis, which is still driven by experimental techniques that rely on data collected from multiple tissue samples. (Asp et al., Cell 2019; 179: 1647-1660) Combining the single-cell genetic manipulation and incisional biopsy features of the probe 400 for a sequential spatially resolved genetic manipulation, and overall omics sampling for multiplexed single-cells, is indeed a groundbreaking addition to Human Cell Atlas mission. (Regev et al., Elife 2017; 6: e27041) This is one of the most ambitious genomics projects which aims to map all human cells. The probe 400 doesn't only offer it a time-based reference frame, but also provides a means of creating cell atlas data based on the response of samples to artificial genetic stimulation.

HeLa and MCF-7 cells (ATCC, VA, USA) were cultured in sterile Petri Dishes (Thermo Scientific) in Dulbecco's modified Eagle medium (DMEM) supplemented with 10% fetal bovine serum (FBS) at 37° C. and 5% CO₂ in humidified air. Cells were seeded on Indium Tin Oxide (ITO)coated glass slides with resistivity of 15-25 Ω/sq (Sigma Aldrich, Saint-Louis, USA) at a density of 500,000 cells per ml for 24 h before the experiments. Primary Human Foreskin Fibroblast (HFF) and Mouse Embryonic Fibroblast (MEFs) (PromoCell GmbH, Heidelberg, Germany) were cultured in the same media and conditions but seeded on the ITO coated glass slide at density of 150,000 cells per ml for 24 h before the experiments. Prior to the transfection and biopsy experiments, the cell culture media was replaced with a low conductivity electroporation buffer (EB). EB used for the transfection and biopsies experiments was prepared by dissolving 95 g sucrose, 0.1 g dextrose, 3 ml 1M HEPES solution in 950 ml DI water. The pH was adjusted to 7.4 and conductivity to 140 mS/m with 1 M NaOH and DPBS respectively. The osmolarity of the media used for electroporation was adjusted to 290 mmol/kg while that for biopsies was adjusted to 190 mm/kg.

Designs of the MMFP, chip-to-holder adapter, and probe holder were created on a commercial CAD software (SolidWorks). The MMFP was fabricated in one step using the Nanoscribe Photonics Professional GT system (Nanoscribe GmbH, Eggenstein-Leopoldshafen, Germany) with the commercially available IP-S resin (Nanoscribe GmbH, Eggenstein-Leopoldshafen, Germany). Upon printing, the MMFP was soaked in IP-S developer (Nanoscribe GmbH, Eggenstein-Leopoldshafen, Germany) for 24 h, rinsed thoroughly with ethanol and dried with an air gun. The chip-to-holder adapter was fabricated in one step using a digital light processing (DLP) projector-based stereolithographic (SLA) 3D printer with a XY resolution of 25 μm (Solus DLP, Junction3D, Santa Clarita, Calif., USA). The through holes in the chip-to-holder adapter were fabricated with a 760 μm diameter to accommodate in-house machined metallic tubing (pins) with a 720 μm external diameter. To ensure sterility, the chip-to-holder adapter and pins were subsequently soaked in ethanol for 12 hours and rinsed with DI water. The probe holder was also fabricated in one step using the VeroYellow material (Stratasys Rehovot, Israel) on a PolyJet 3D Printer (Stratasys J750 Stratasys Rehovot, Israel). The MMFP, pins, and chip-to-holder adapter were assembled and affixed together by glue. The assembly was then made conductive by sputtering gold on the entire surface (Cressington Sputter Coater, Ted Peller, CA, USA) until a surface resistivity of 1 Ω sq.⁻¹ was attained. Assembly of these with the probe holder, to form the complete MMFP setup, was achieved by the twist lock mechanism. ITO coated glass slide (8-12 Ω sq.⁻¹, Sigma Aldrich, Saint-Louis, USA) were used as the counter electrodes and electrical wires were affixed to them using silver conductive epoxy (M.G Chemicals Limited, Ontario, Canada). Electrical contacts to the electrical wires and the MMFP were made by clippings. This assembly was mounted on an XYZ micro controller and positioned atop the objective of an inverted microscope.

The 3D finite element models of the MMFP's HFC were developed using a commercial finite element solver (COMSOL Multiphysics® v.5.2. COMSOL AB, Stockholm, Sweden). Numerical solutions were acquired by iteratively solving for the coupled Navier-Stokes and convection-diffusion equations. A similar iterative solver was used to compute the continuity equation for the electrical potential. MMFPs were modeled as circular surfaces with inlets and outlets apertures, with humps on the tip representing the electrode. The injected and immersion fluids were considered as water with a density of 998.2 kg m⁻³ and a dynamic viscosity of 0.001 Nsm⁻².

The finite element model used as validation of the correlation between vertical and horizontal displacement of the probe setup was built using a solid mechanics model with linear elastic material properties. The contact between the MMFP and the bottom substrate were modelled as a friction contact with a coefficient of friction (CF) 0.18.

The MMFP assembly was mounted on an XYZ micro controller (X-LRM, Zaber, Vancouver, Canada) and positioned atop of the objective of an inverted microscope (Epi-fluorescence inverted ECLIPSE Ti microscope (NIKON, Tokyo, Japan)). The ITO coated glass slide with cells cultured on a portion for 24 hrs. were placed on the microscope stage—sandwiched between the objective and the MMFP. The Z axis of the XYZ microcontroller was then used to slowly approach a glass slide portion without any cells and monitoring is performed using a combination of aDS-Qi2 camera (NIKON) and an image recognition tool. A horizontal displacement of the MMFP is indicative of contact between the probe and the glass slide. Real-time measurement of this displacement for 5 secs provides the calibration tool with an input to deduce the upward vertical pull required to zero the tip-substrate gap, based on the correlation deduced and validated. Upon zeroing, a 20 μm tip-substrate gap is created and the probe is positioned above the target cell using the X and Y axes.

Propidium Iodide (eBioscience, California, United States) and 240 kDA FITC dextran (Sigma-Aldrich, Mo., USA) were dissolved in the low conductivity EB to final concentrations of 25 ng/ml and 10 mg/ml respectively. Green fluorescein sodium salt (C20H10Na2O5) (Sigma Aldrich, Mo., USA) was dissolved in the PI-EB solution (final concentration of 1 mg/ml) to aid visibility. The reagent was loaded in syringes, connected to the metallic pins of the MMFP assembly via tubing, and controlled by neMESYS high precision syringe pump (CETONI, Korbuβen, Germany) to form the HFC. Cell culture media were replaced with EB by gentle rinsing and the whole culture substrate was positioned on the stage of the microscope. Electric potential was created and controlled by a function generator (Keysight 33500B Series, California, US) to produce 1 sec DC electric pulses with 50 ms pulse width and 10 Hz frequency during the 3 min cell exposure to the HFC.

The MMFP's scanning operation was performed by programming (C #) the XY stage movements in the Zaber scripting environment. The X and Y stages were moved individually for horizontal and vertical lines respectively, and simultaneously for slanted lines. Spacing between each letter was achieved by rapid movement of the stages (˜29 mm/s), from the end of one letter to the beginning of the other letter.

Cell viability after the electroporation procedure was confirmed by employing a Live/Dead viability/cytotoxicity kit for mammalian cells (Invitrogen, Thermo Scientific, MA, US). For this, cells that underwent electroporation were incubated in a solution of calcein AM (0.5 μL of 4 mM solution per mL of culture media) and epithidium homodimer-1 (2 μL of 2 mM solution per mL of cell media) for 10 minutes. After incubation, the Live/Dead viability solution was removed by gently washing with fresh growth media. FITC and TRITC images were obtained with a ×20 objective and fluorescently labelled Live/Dead cells were manually counted on ImageJ (GNU Library).

pCMV-GFP (Addgene plasmid #11153) and mCherry2-C1 (Addgene plasmid #54563) plasmids were obtained in bacteria as agar stab (Addgene, MA, USA). Individual bacteria colony populations were isolated from the stock by streaking on Luria Broth (LB) agar plates and incubating at 37° C. for 12-18 hrs. LB agar plates were prepared from the powder LB Broth (Miller, Sigma-Aldrich, Saint-Louis, USA) by dissolving 25 g broth powder and 15 g bacteriological agar in 1 L ultrapure water. Ampicillin (100 μg/mL) and Kanamycin (50 μg/mL) are also dissolved in the LB broth/agar mixture for pCMV-GFP and mCherry2-C1 containing bacteria, respectively. Individual colonies of the bacteria were grown by incubating a swab in 25 mL liquid LB media at 37° C. for 12-18 hrs. on a shaker. Plasmids were extracted from the bacteria using the QIAprep Spin Miniprep Kit (Qiagen, Hilden Germany), according to the kit's protocol. Quantification of the extracted plasm ids were carried out on the NanoDrop (ThermoScientific, MA, US). Purified plasm ids were suspended in EP buffer to a final concentration of 5 ng/ml, loaded into syringes and injected through the inlet aperture of the MMFP. Growth media of the cells cultured on the ITO coated glass slide is replaced with EB and placed on microscope's stage. A 2V amplitude DC electric pulse (50 ms pulse) is introduced for 1 sec and the target cell is confined by the HFC of transfection reagent for 3 minutes. After electroporation, EP buffer is replaced with growth media and the cells are returned back to the incubator for 24 hrs. Estimation of the transfection efficiency is based on manual counting of the fluorescently labeled cells and the total number of cells covered by the HFC footprint.

To visualize cytoplasmic extraction, cells were pre-labelled with calcein-AM (Invitrogen, Thermo Scientific, MA, US). Growth media was replaced with EB and the cells were positioned on the microscope's stage. The MMFP was positioned 20 μm above the target cell and a 100 kHz AC electric signal was activated for 1 sec to electroporate/lyse the cell, while the HFC of EP buffer was activated for 1 minute to extract the content. After extraction, the MMFP was pulled up by the Z axis controller and the extracted content was transferred into RNase-free micro centrifuge tubes as a 10 μL solution. As positive control, 10 cells were isolated by serial dilution and lysed in 9 μL of Single-cell Lysis Solution (ThermoScientific, MA, US). Lysing was allowed to be incubated at room temperature for 5 minutes after which lying was stopped using 1 μL of Single-cell Stop (ThermoScientific, MA, US).

To enable visibility, cells cultured on the ITO coated glass slide were stained with CellTracker® Red CMTPX Dye with a final concentration of 10 μM. To aid visibility of the HFC, green fluorescein sodium salt (C20H10Na2O5) (Sigma Aldrich, Mo., USA) was dissolved in Trypsin-EDTA solution (Sigma-Aldrich) with a final concentration of 1 mg/mL. The cells were placed on the microscope's stage and the MMFP was positioned 20 μm above the target cell. Simultaneous activation of the HFC and AC electric signal (10 MHz) effected enzymatic cell dissociation while increasing surrounding media temperature. The AC signal was induced by a function generator (Agilent 33521A, Agilent Scientific Instruments, California, USA) and a high-power amplifier (ZHL-5 W-1, Mini Circuits New York, USA) was used to attain voltages greater than 10 V. Upon detachment of the cell, the inlet fluid flow is stopped to allow the suction flow to further dilute trypsin concentration in collected cell suspension. The dissociated cell is then transferred to RNase-free micro centrifuge tubes where they are lysed with Single-cell Lysis Solution (ThermoScientific, MA, US). Voltage dependent temperature measurements were collected by pointing an infrared camera (Flir, Tester UK) at the target area.

Reverse transcription (cDNA synthesis) and pre-amplification of the collected RNAs were performed using a PCR thermal cycler (Labnet MultiGene, Sigma Aldrich, Mo., USA). The reagents and protocol used are contained in the Ambion Single-cell-to-CT Kit for qRT-PCR (ThermoScientific, MA, US). Prior to pre-amplification, 1 μL Single-cell DNase was added to the extracted RNAs. To this, a mixture of Single-cell VILO RT Mix (3 μL) and Single-cell SuperScript RT (1.5 μL) were added. Following, the mixture was placed in the thermal cycler for initial priming at 25° C. for 10 minutes, reverse transcription at 42° C. for 60 minutes, and reverse transcriptase inactivation at 85° C. for 5 minutes.

The pre-amplification mixture was prepared by adding 0.2×TaqMan Gene Expression Assay with RNase-P primer (6 μL) and Single-cell PreAmp Mix (5 μL) to the reverse transcribed sample. Following, the mixture was held in the thermal cycler for enzymatic activation at 95° C. for 10 minutes, passed through 14 cycles of denaturation at 95° C. for 15 secs, annealing/extension at 60° C. for 4 minutes, and enzymatic deactivation at 99° C. for 10 minutes.

All qPCR amplification experiments were carried out using a StepOnePlus Real-Time PCR System (Applied Biosystems, CA, US) in MicroAmp Fast Optical 96-Well reaction plates (Applied Biosystems, CA, US). The TaqMan Probes and Primers were obtained commercially from Applied Biosystems. 4 μL pre-amplified cDNA was first transferred into the 96-Well reaction plates that contained a mixture of Taqman Gene Expression Master Mix (10 μL), TaqMan Gene Expression Assay (1 μL) and Nuclease-free water (5 μL). After initial incubation at 50° C. for 2 minutes and enzymatic activation at 95° C. for 10 minutes, 50 PCR cycles were performed (denaturation at 95° C. for 5 s, and annealing/extension at 60° C. for 1 minute). Fluorescence data were recorded at the end of each annealing/extension step.

The following references are each hereby incorporated herein by reference in their entirety:
Brimmo, A. T., Menachery, A., Sukumar, P., Qasaimeh, M. A., Noncontact Multiphysics Probe for Spatiotemporal Resolved Single-Cell Manipulation and Analyses. Small 2021, 17, 2100801. https://doi.org/10.1002/sm11.202100801
Wang D, Bodovitz S. Single cell analysis: the new frontier in ‘omics’. Trends Biotechnol 2010; 28: 281-290.
Klepárnik K, Foret F. Recent advances in the development of single cell analysis—A review. Anal Chim Acta 2013; 800: 12-21.
Bedard P L, Hansen A R, Ratain M J, Siu L L. Tumour heterogeneity in the clinic. Nature 2013; 501: 355-364.
Peng A, Mao X, Zhong J, Fan S, Hu Y. Single-Cell Multi-Omics and Its Prospective Application in Cancer Biology. Proteomics 2020; : 1900271.
Hu P, Zhang W, Xin H, Deng G. Single cell isolation and analysis. Front cell Dev Biol 2016; 4: 116.
Gross A, Schoendube J, Zimmermann S, Steeb M, Zengerle R, Koltay P. Technologies for single-cell isolation. Int J Mol Sci 2015; 16: 16897-16919.
Prakadan S M, Shalek A K, Weitz D A. Scaling by shrinking: empowering single-cell‘omics’ with microfluidic devices. Nat Rev Genet 2017; 18: 345-361.
Xu X, Wang J, Wu L, Guo J, Song Y, Tian T et al. Microfluidic Single-Cell Omics Analysis. Small 2020; 16: 1903905.
S. C. Bürgel, C. Escobedo, N. Haandbæk, A. Hierlemann, Sens. Actuators, B 2015, 210, 82.
Yin H, Marshall D. Microfluidics for single cell analysis. Curr Opin Biotechnol 2012; 23: 110-119.
H. H. Wang, F. J. Isaacs, P. A. Carr, Z. Z. Sun, G. Xu, C. R. Forest, G. M. Church, Nature 2009, 460, 894.
Joensson H N, Andersson Svahn H. Droplet microfluidics—A tool for single-cell analysis. Angew Chemie Int Ed 2012; 51: 12176-12192.
Rosenthal A, Macdonald A, Voldman J. Cell patterning chip for controlling the stem cell microenvironment. Biomaterials 2007; 28: 3208-3216.
Wheeler A R, Throndset W R, Whelan R J, Leach A M, Zare R N, Liao Y H et al. Microfluidic device for single-cell analysis. Anal Chem 2003; 75: 3581-3586.
Qin Y, Wu L, Schneider T, Yen G S, Wang J, Xu S et al. A Self-Digitization Dielectrophoretic (SD-DEP) Chip for High-Efficiency Single-Cell Capture, On-Demand Compartmentalization, and Downstream Nucleic Acid Analysis. Angew Chemie Int Ed 2018; 57: 11378-11383.
H. P. Schwan, Biological Effects and Dosimetry of Nonionizing Radiation, Springer, Boston, Mass. 1983, pp. 213-231.
Li M, Anand R K. High-throughput selective capture of single circulating tumor cells by dielectrophoresis at a wireless electrode array. J Am Chem Soc 2017; 139: 8950-8959.
Buenrostro J D, Wu B, Litzenburger U M, Ruff D, Gonzales M L, Snyder M P et al. Single-cell chromatin accessibility reveals principles of regulatory variation. Nature 2015; 523: 486-490.
Tirosh I, Izar B, Prakadan S M, Wadsworth M H, Treacy D, Trombetta J J et al. Dissecting the multicellular ecosystem of metastatic melanoma by single-cell RNA-seq. Science (80-) 2016; 352: 189-196.
B. W. Solnestam, H. Stranneheim, J. Hällman, M. Käller, E. Lundberg, J. Lundeberg, P. Akan, BMC Genomics 2012, 13, 1.
Treutlein B, Brownfield D G, Wu A R, Neff N F, Mantalas G L, Espinoza F H et al. Reconstructing lineage hierarchies of the distal lung epithelium using single-cell RNA-seq. Nature 2014; 509: 371-375.
A. T. Brimmo, M. A. Qasaimeh, RSC Adv. 2017, 7, 5120.
Lawson D A, Bhakta N R, Kessenbrock K, Prummel K D, Yu Y, Takai K et al. Single-cell analysis reveals a stem-cell program in human metastatic breast cancer cells. Nature 2015; 526: 131-135.
Brito I L, Yilmaz S, Huang K, Xu L, Jupiter S D, Jenkins A P et al. Mobile genes in the human microbiome are structured from global to individual scales. Nature 2016; 535: 435-439.
Tanay A, Regev A. Scaling single-cell genomics from phenomenology to mechanism. Nature 2017; 541: 331-338.
Wagner A, Regev A, Yosef N. Revealing the vectors of cellular identity with single-cell genomics. Nat Biotechnol 2016; 34: 1145-1160.
Kafri R, Levy J, Ginzberg M B, Oh S, Lahav G, Kirschner M W. Dynamics extracted from fixed cells reveal feedback linking cell growth to cell cycle. Nature 2013; 494: 480-483.
Bar-Joseph Z, Siegfried Z, Brandeis M, Brors B, Lu Y, Eils R et al. Genome-wide transcriptional analysis of the human cell cycle identifies genes differentially regulated in normal and cancer cells. Proc Natl Acad Sci 2008; 105: 955-960.
Lu Y, Mahony S, Benos P V, Rosenfeld R, Simon I, Breeden L L et al. Combined analysis reveals a core set of cycling genes. Genome Biol 2007; 8: R146.
Fattore L, Ruggiero C F, Liguoro D, Mancini R, Ciliberto G. Single cell analysis to dissect molecular heterogeneity and disease evolution in metastatic melanoma. Cell Death Dis 2019; 10: 1-12.
Yu H, Kortylewski M, Pardoll D. Crosstalk between cancer and immune cells: role of STAT3 in the tumour microenvironment. Nat Rev Immunol 2007; 7: 41-51.
Yu R, Ying Y, Gao R, Long Y. Confined nanopipette sensing: from single molecules, single nanoparticles, to single cells. Angew Chemie Int Ed 2019; 58: 3706-3714.
Guillaume-Gentil O, Potthoff E, Ossola D, Franz C M, Zambelli T, Vorholt J A. Force-controlled manipulation of single cells: from AFM to FluidFM. Trends Biotechnol 2014; 32: 381-388.
Nadappuram B P, Cadinu P, Barik A, Ainscough A J, Devine M J, Kang M et al. Nanoscale tweezers for single-cell biopsies. Nat Nanotechnol 2019; 14: 80-88.
Guillaume-Gentil O, Grindberg R V, Kooger R, Dorwling-Carter L, Martinez V, Ossola D et al. Tunable single-cell extraction for molecular analyses. Cell 2016; 166: 506-516.
Tsong T Y. Electroporation of cell membranes. In: Electroporation and electrofusion in cell biology. Springer, 1989, pp 149-163.
C. A. Jordan, E. Neumann, A. E. Sowers, Electroporation and Electrofusion in Cell Biology, Springer Science & Business Media, Berlin/Heidelberg, Germany 2013.
Juncker D, Schmid H, Delamarche E. Multipurpose microfluidic probe. Nat Mater 2005; 4: 622-627.
Anscombe N. Direct laser writing. Nat Photonics 2010; 4: 22-23.
P. Mukherjee, E. J. Berns, C. A. Patino, E. Hakim Moully, L. Chang, S. S. P. Nathamgari, J. A. Kessler, M. Mrksich, H. D. Espinosa, Small 2020, 16, 2000584.
Safavieh M, Qasaimeh M A, Vakil A, Juncker D, Gervais T. Two-aperture microfluidic probes as flow dipole: Theory and applications. Sci Rep 2015; 5: 11943.
Goyette P A, Boulais E, Normandeau F, Laberge G, Juncker D, Gervais T. Microfluidic multipoles theory and applications. Nat Commun 2019; 10: 1781.
Qasaimeh M A, Gervais T, Juncker D. Microfluidic quadrupole and floating concentration gradient. Nat Commun 2011; 2: 464.
Qasaimeh M A, Ricoult S G, Juncker D. Microfluidic probes for use in life sciences and medicine. Lab Chip 2013; 13: 40-50.
K. KinositaJr, H. Itoh, S. Ishiwata, K. Hirano, T. Nishizaka, T. Hayakawa, J. Cell Biol. 1991, 115, 67.
Brimmo A T, Qasaimeh M A. Implementation and Applications of Microfluidic Quadrupoles. Open-sp Microfluid Concepts, Implementations, Appl 2018.
Delamarche E, Kaigala G V. Open-space microfluidics: concepts, implementations, applications. John Wiley & Sons, 2018.
Brimmo A, Goyette P A, Alnemari R, Gervais T, Qasaimeh M A. 3D Printed Microfluidic Probes. Sci Rep 2018; 8: 10995.
Beare W G, Bowden F P. Physical properties of surfaces I-Kinetic friction. Philos Trans R Soc London Ser A, Math Phys Sci 1935; 234: 329-354.
Pohl H A, Crane J S. Dielectrophoresis of cells. Biophys J 1971; 11: 711-727.
Ludwig A, Kretzmer G, Schügerl K. Determination of a “critical shear stress level” applied to adherent mammalian cells. Enzyme Microb Technol 1992; 14: 209-213.
Henriques J F, Caseiro R, Martins P, Batista J. High-Speed Tracking with Kernelized Correlation Filters. IEEE Trans Pattern Anal Mach Intell 2015; 37: 583-596.
Weaver J C. Electroporation of cells and tissues. IEEE Trans plasma Sci 2000; 28: 24-33.
Jordan C A, Neumann E, Sowers A E. Electroporation and electrofusion in cell biology. Springer Science & Business Media, 2013.
Weaver J C. Electroporation theory. In: Plant cell electroporation and electrofusion protocols. Springer, 1995, pp 3-28.
A. T. Brimmo, M. A. Qasaimeh, IEEE Nanotechnol. Mag. 2017, 11, 20.
Weaver J C, Chizmadzhev Y A. Theory of electroporation: a review. Bioelectrochemistry Bioenerg 1996; 41: 135-160.
Jordan M, Wurm F. Transfection of adherent and suspended cells by calcium phosphate. Methods 2004; 33: 136-143.
Weaver J C, Harrison G I, Bliss J G, Mourant J R, Powell K T. Electroporation: high frequency of occurrence of a transient high-permeability state in erythrocytes and intact yeast. FEBS Lett 1988; 229: 30-34.
G. Serša, M. Čemažar, D. Šemrov, D. Miklavčič, Bioelectrochem. Bioenerg. 1996, 39, 61.
Canatella P J, Karr J F, Petros J A, Prausnitz M R. Quantitative study of electroporation-mediated molecular uptake and cell viability. Biophys J 2001; 80: 755-764.
Douglas K L. Toward development of artificial viruses for gene therapy: a comparative evaluation of viral and non-viral transfection. Biotechnol Prog 2008; 24: 871-883.
Matsuda T, Cepko C L. Electroporation and RNA interference in the rodent retina in vivo and in vitro. Proc Natl Acad Sci 2004; 101: 16-22.
Haas K, Sin W-C, Javaherian A, Li Z, Cline H T. Single-cell electroporationfor gene transfer in vivo. Neuron 2001; 29: 583-591.
Judkewitz B, Rizzi M, Kitamura K, Häusser M. Targeted single-cell electroporation of mammalian neurons in vivo. Nat Protoc 2009; 4: 862.
Olofsson J, Nolkrantz K, Ryttsén F, Lambie B A, Weber S G, Orwar O. Single-cell electroporation. Curr Opin Biotechnol 2003; 14: 29-34.
Brimmo A T, Menachery A, Qasaimeh M A. Microelectrofluidic probe for sequential cell separation and patterning. Lab Chip 2019; 19: 4052-4063.
Abdelmoez M N, lida K, Oguchi Y, Nishikii H, Yokokawa R, Kotera H et al. SINC-seq: correlation of transient gene expressions between nucleus and cytoplasm reflects single-cell physiology. Genome Biol 2018; 19: 1-11.
Rick W. Trypsin. In: Methods of enzymatic analysis. Elsevier, 1974, pp 1013-1024.
Menachery A, Pethig R. Controlling cell destruction using dielectrophoretic forces. IEE Proc—Nanobiotechnology 2005; 152: 145-149.
Svec D, Tichopad A, Novosadova V, Pfaffl M W, Kubista M. How good is a PCR efficiency estimate: Recommendations for precise and robust qPCR efficiency assessments. Biomol Detect Quantif 2015; 3: 9-16.
Asp M, Giacomello S, Larsson L, Wu C, Fürth D, Qian X et al. A spatiotemporal organ-wide gene expression and cell atlas of the developing human heart. Cell 2019; 179: 1647-1660.
Regev A, Teichmann S A, Lander E S, Amit I, Benoist C, Birney E et al. Science forum: the human cell atlas. Elife 2017; 6: e27041.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A non-contact cell manipulation system, comprising:
a supporting member;
a micro-positioner;
a probe holder having a proximal end and a distal end, the proximal end connected to the micro-positioner;
a probe adapter removably connected to the distal end of the probe holder; and
a non-contact multiphysics probe fluidly and electrically connected to the probe adapter, wherein the probe includes at least one electrode, at least one aperture, and wherein the probe is configured to utilize electropermealization and electroheating in combination with hydrodynamic flow confinement to perform non-contact cell manipulation.

2. The system of claim 1, wherein a subset of the at least one electrode is configured as a counter-electrode.

3. The system of claim 1, wherein the probe is a 3D printed part.

4. The system of claim 1, further comprising a cell culture glass slide coated in an ITO substrate.

5. The system of claim 4, wherein the electrode and the ITO coated substrate form a pin-plate electrode.

6. A non-contact multiphysics probe, comprising:
at least one aperture; and
at least one electrode, wherein the electrode and aperture are configured to perform electropermealization and electroheating in combination with hydrodynamic flow confinement.

7. The probe of claim 6, wherein the at least one electrode has a radius of 0.1-100 μm.

8. The probe of claim 6, wherein the at least one electrode has a height of 0.1-1000 μm.

9. The probe of claim 6, wherein the at least one electrode is at least one shape selected from the group consisting of a dome shape, a pin shape, a conical shape, a spiked shape, a cylindrical shape, a suspended shape, and a pyramidal shape.

10. The probe of claim 6, wherein the probe comprises at least one inlet aperture and at least one outlet aperture.

11. The probe of claim 10, wherein the at least one inlet aperture has a radius of 0.1-1000 μm, and the at least one outlet aperture has a radius of 0.1-1000 μm.

12. The probe of claim 10, wherein the at least one inlet aperture and the at least one outlet aperture are spaced at least 1 μm center-to-center apart.

13. The probe of claim 12, wherein the at least one electrode is positioned centrally between the at least one inlet aperture and the at least one outlet aperture.

14. A non-contact cell manipulation method, comprising:
providing a non-contact cell manipulation probe;
positioning the probe above a cell to be manipulated;
applying an electric field via an electrode located on the probe; and
applying hydrodynamic flow confinement to manipulate the cell.

15. The method of claim 14, wherein the electric field and hydrodynamic flow confinement are applied while the cell is still within its original tissue organization.

16. The method of claim 14, wherein the electric field is pulsed.

17. The method of claim 14, wherein the manipulation performed is a single-cell biopsy operation.

18. The method of claim 14, wherein the manipulation performed is a heat assisted single-cell tweezer operation.

19. The method of claim 14, wherein prior to positioning the probe over the cell, the probe is zeroed based on horizontal displacement when the probe contacts a substrate.

20. The method of claim 14, further comprising positioning the probe 0.1-1000 μm above a substrate holding the cell.
